(19) United States
(12) Patent Application Publication
(10) Pub. No.: US 2013/0078657 A1
(43) Pub. Date: Mar. 28, 2013

(54) HYPERTHERMIA AUGMENTED IN-VITRO IMMUNE RECOGNITION

(71) Applicants: Morten Ruhwald, Copenhagen V (DK); Jesper Eugen-Olsen, Hellerup (DK); Pernille Ravn, Frederiksberg (DK); Martine Grosos Aabye, Copenhagen V (DK)

(72) Inventors: Morten Ruhwald, Copenhagen V (DK); Jesper Eugen-Olsen, Hellerup (DK); Pernille Ravn, Frederiksberg (DK); Martine Grosos Aabye, Copenhagen V (DK)

(73) Assignee: Hvidovre Hospital, Hvidovre (DK)

(21) Appl. No.: 13/695,219

(22) PCT Filed: May 4, 2011
(86) PCT No.: PCT/DK2011/000041

§ 371 Date: Dec. 14, 2012

(30) Foreign Application Priority Data
May 4, 2010 (EP) 10161843.7

(51) Int. Cl. G01N 33/53

(52) U.S. Cl. CPC G01N 33/5306
USPC 435/7.94; 435/7.92

(57) ABSTRACT

The present invention relates to a method for generation of a test-antigen specific cell-mediated immune response by incubating at hyperthermic conditions and, more particularly, a method for generation of a test-antigen specific cell-mediated immune response by incubating at hyperthermic conditions and optionally adding IL-7 and/or blocking IL-10. Even more particularly, the present invention provides a method for generating a cell-mediated response to an antigen using whole blood or other suitable bio-logical samples. The method is useful in for immune diagnosis of many infectious diseases, as a marker of immunocompetence, and for detection of T-cell responses to non-self antigens (i.e. infections and vaccines).

### FIELD OF THE INVENTION

The present invention relates to a general method for generation of a test-antigen specific cell-mediated immune response by incubating at hyperthermic conditions and, more particularly, a method for generation of a test-antigen specific cell-mediated immune response by incubating at hyperthermic conditions and optionally adding IL-7 and/or blocking IL-10. Even more particularly, the present invention provides a method for generating a cell-mediated response to an antigen using whole blood or other suitable biological samples. The method is useful in therapeutic and diagnostic protocols for human, livestock and veterinary as well as wild life applications.

Measurement of cell-mediated immune responses is important for immune diagnosis of many infectious diseases, as a marker of immunocompetence, and for detection of T-cell responses to non-self antigens (i.e. infections and vaccines).

The present invention provides a method for generating and/or evaluating a test-antigen specific cell-mediated immune response in a mammal by incubating, in the presence of at least one antigen, a sample from the mammal comprising cells of the immune system capable of elicting an immune response, at hyperthermic conditions. The method may comprise a supplementary step comprising addition of at least one immune modulator such as IL-7 and/or antibodies binding IL-10. Production of at least one immune signaling molecule such as IP-10 and/or IFN-γ is then detected. The presence or level of immune signaling molecules is then indicative of the level of cell-mediated responsiveness of the subject.

### BACKGROUND OF THE INVENTION

Hyperthermic Incubation

The usefulness of fever-like temperatures in immunological methods in general is quite controversial.

Few studies have been published on the effect of differential incubation temperature in immuno-diagnostic methods and these report decreased IFN-γ production at 37.5° C. compared to lower temperatures (around 21° C.), when incubating whole blood from cattle with mitogen (Waters et al: 2007, Robbe-Austerman et al. 2006). Nevertheless, Interferon (IFN)-γ release assay (IGRA) manufacturers recommend an incubation temperature of 37° C. (www.cellestis.com), which seems to give fairly good IFN-γ responses, although to our knowledge data supporting the choice of incubation temperature has not been published.

Studies on how increasing temperature affects cells of the immune system in-vitro are presently not conclusive. Results from studies where cells of the immune system have been incubated at hyperthermic temperatures are conflicting to whether or not temperatures up to 40-41° Celsius augment immune responsiveness.

Some studies have shown an improvement of the immune function at hyperthermic incubation. A study by Basu et al. use purified and “heat shocked” (6 h incubation at 41° Celsius) murine dendritic cells (DCs) loaded with the OVA specific SIINFEKL peptide and compare the effects of “heat shocked” DCs to normal DCs in interaction with a T cell line specific for the SIINFEKL peptide. The study demonstrated that the “heat shocked” DCs were able to augment the production of IFN-γ up to 3-fold compared to normal DCs (Basu et al 2003).

Another study demonstrated that when exposing human PBMCs to 40° or 41° Celsius for 6 hours followed by stimulation with mitogen or tetanous toxoid at 37° C. increased the number of T cells producing IFN-γ, and increased the proliferative responses. Adding monoclonal antibodies to MHC class II abrogated the effects completely (Huang et al. 1996). Similar effects have been demonstrated for stimulation with PPD (Kappel et al. 1991). It seems that hyperthermia mediates the effects through upregulation of MHC class II and of costimulatory molecules like the B7 family members CD80/86 on the antigen presenting cells.

However none of these studies show that incubation at higher temperatures i.e. hyperthermic conditions have an effect on disease-specific antigen recognition.

The closest prior art is a study by Schiller et al. 2009, which studies disease-specific antigen recognition. In this study whole blood from M. tuberculosis infected cattle was incubated at various temperatures 25°, 29°, 33° and 39° Celsius and IFN-γ responses to the M. tuberculosis specific antigens ESAT6 and CFP10 were studied. However, the authors do not observe a difference in IFN-γ responsiveness between 33° and 39° Celsius. (The samples were not measured at 37° C.)(Schiller et al. 2009). This is in conflict with the teachings of the present invention where we demonstrate how incubation at hyperthermic conditions augment a test-antigen specific cell-mediated immune response.

IL-7 & Anti-IL-10

It is desirable to further improve a test antigen specific cell-mediated immune response. This can be done by adding an immuno-modulator during the incubation step. We have exemplified this improvement in the present invention by adding IL-7 and/or neutralizing antibodies binding IL-10.

Feske et al. have studied the effects of adding IL-7 to ESAT6/CFP10 stimulated blood from TB patients and demonstrated an increase in the production of IFN-γ (Feske et al. 2008). However, it has never been tested or suggested that it is possible to improve biomarker responses by the combination of hyperthermia and IL-7.

Denis et al have demonstrated that adding monoclonal antibodies to IL-10 during culture of bovine blood in the presence of ESAT6/CFP10 improves the IFN-γ response and sensitivity of this TB test [Denis et al 2007]. Improving biomarker responses by the combination of hyperthermia and blockage of IL-10 is new and has not been proposed or tested.

Tuberculosis

The discovery of mycobacterium tuberculosis (MTB)-specific immunodominant antigens has led to a significant new avenue for the diagnosis of tuberculosis (TB). Early studies showed that a test that assayed the in-vitro production of interferon gamma (IFN-γ) by T cells in response to defined MTB antigens had potential to replace the Tuberculin Skin Test (TST). Around the same time, a major advance was the discovery of the highly immunogenic antigens, early secreted antigenic target 6 (ESAT-6), culture filtrate protein 10 (CFP-10) and TB7.7 that improved specificity significantly. These antigens are encoded within the region of difference 1 (RD1) and RD11 of the pathogen and are consequently absent from all Bacille Calmette Guerin (BCG) vaccine strains and most non-tuberculous mycobacteria (exceptions include Mycobacterium kansasii, Mycobacterium marinum, and Mycobacterium szulgai). IFN-γ responses to overlapping peptides of the RD1 and RD11 encoded antigens ESAT-6, CFP-10, TB7.7 form the basis for the detection of MTB infection in two licensed and commercially available tests.

QuantiFERON-TB Gold (Cellestis Limited, Carnegie, Victoria, Australia), a whole blood enzyme-linked immunoassay (ELISA) has European CE mark and American Food and Drug Administration (FDA) approval for the detection of both latent TB infection and disease.

T-SPOT.TB (Oxford Immunotec, Oxford, UK), an enzyme-linked immunospot assay (ELISPOT) that uses peripheral blood mononuclear cells has European CE mark approval and was approved for use in Canada in 2005. T-SPOT.TB only uses ESAT-6 and CFP10.

Unfortunately sensitivity of these tests is impaired in immunocompromised individuals (such as HIV-infected individuals or patients receiving immunosuppressing medication). Thus, it is desirable to develop more sensitive tests that allow for diagnosis of these patients who have otherwise inadequate immune responses to antigens. This will reduce the number of false negative test results and indeterminate test results thus improving sensitivity and cost-effectiveness of immuno-diagnostic tests.

### SUMMARY OF THE INVENTION

The present invention provides a method for augmentation of a test-antigen specific cell-mediated immune response. This method enables better disease-specific antigen recognition.

One aspect of the invention relates to the use of increased temperature during in-vitro incubation of a biological sample with at least one antigen. The incubation at increased temperature leads to an increase in the test-antigen specific cell-mediated immune response compared to incubation at 37° C.

Thus, the present invention provides a method comprising the steps of incubating 30 a biological sample comprising cells of the immune system capable of generating a cell-mediated immune response with at least one test-antigen at hyperthermic conditions such as temperatures between 38 to 42° C. The incubation at hyperthermic conditions augments the test-antigen specific immune response when compared to a reference level obtained by incubation under normal thermic conditions of 37° C.

The present invention also relates to a method further comprising the step of adding at least one immune modulator such as IL-7 and/or anti-IL-10 to improve the test-antigen specific cell-mediated immune response.

One aspect of the invention relates to an optimized method for determining the ability or capacity of a subject to mount a test-antigen specific cell-mediated immune response. The method is based on measuring production of one or more immune signalling molecules from cells of the immune system in response to antigenic stimulation. The immune signalling molecules may be detected using ligands such as antibodies specific for the immune signalling molecules or by determining the level of expression of genes encoding the immune signalling molecules.

Another aspect of the invention relates to an optimized method to determine the immunogenicity i.e. potential or capacity of an antigen to generate a cell mediated immune response. This method to monitor cellular immune responses is one prerequisite for rational development of vaccines.

The present invention provides, therefore, means to determine the responsiveness of test-antigen specific cell-mediated immune response in a subject and, in turn, provides means for the diagnosis of infectious diseases, pathological conditions, estimation of the level of immunocompetence and the level of T-cell responsiveness to endogenous or exogenous antigens.

The method provided by the invention reduces the number of false negative and indeterminate test results and thus increases sensitivity compared to tests performed at normal incubation temperatures i.e. 37° C. Therefore the method improves testing and diagnosing.

According to the present invention patients/donors with low or weak immune responses under normal incubation conditions can be brought to respond strongly by increasing the incubation temperature, especially in the presence of at least one immune modulator such as IL-7 and/or anti-IL-10. Thereby this invention enable immunological diagnosis of patients with otherwise inadequate immune responses to antigens and it also reduces the number of false negative test results. Thus, the invention improves the sensitivity and cost-effectiveness of immuno-diagnostic tests, importantly also in immuno-compromised individuals. Furthermore, it may play a role in vaccine development and monitoring e.g. for infectious agents and cancer.

### DETAILED DESCRIPTION

The present invention concerns a novel method to augment a test-antigen specific cell-mediated immune response.

It is shown for the first time that cytokine and chemokine responseses are dramatically increased with hyperthermic incubation compared to traditional incubation at 37° Celsius.

The present invention also shows for the first time that the augmented immune recognition at hyperthermic incubation is a phenomenon that applies to immune responses towards antigens that are specific for a disease or a vaccine. It also shows how the augmented immune recognition at hyperthermic incubation can be applied in a diagnostic test or in an assessment of a vaccine response.

Furthermore it is demonstrated for the first time that when adding the survival cytokine IL-7 in concert with antibodies that block the anti-inflammatory cytokine IL-10, the immune response is significantly augmented at both hyperthermic (and also normal) incubation temperature.

One aspect of the invention relates to the use of an increased temperature during incubation in-vitro of a biological sample with at least one antigen. The incubation at increased temperature leads to an increase in a test-antigen specific cell-mediated immune response.

The present invention also relates to the use of immune modulators such as IL-7 and/or anti-IL-10 to improve the test-antigen specific cell-mediated immune response.

The present invention provides, therefore, means to augment a test-antigen specific cell-mediated immune response and, in turn, provides means for improving methods and tests wherein it is desired to determine the presence and/or the level of a test-antigen specific cell-mediated response.

Another aspect of the present invention is a method for measuring the potential or capacity of a subject to mount a test-antigen cell-mediated immune response.

The test-antigen specific immune response can be determined by measuring immune signalling molecule production by cells of the immune system in response to antigen stimulation. The immune signalling molecules may be detected using ligands such as antibodies specific for the immune signalling molecules and/or by measuring the level of expression of genes encoding the immune signalling molecules.

Thus, the present invention provides means for the diagnosis of infectious diseases and/or the presence of immune reactivity towards antigens used in vaccines enabling the monitoring of vaccine efficacy.

The present invention provides therefore a simple method by which immune-assays and other immunological tools can be improved.

One aspect of the invention relates to a method for augmenting a test-antigen specific cell-mediated response. The method is based on incubating a sample comprising cells of the immune system capable of generating a cell-mediated immune response with at least one antigen at hyperthermic conditions.

Another aspect of the invention relates to a method to augment a test-antigen specific cell-mediated response based on incubating a sample comprising cells of the immune system capable of generating a cell-mediated immune response with at least one antigen at hyperthermic conditions and in the presence of IL-7.

A third aspect of the invention relates to a method to augment a test-antigen specific cell-mediated response based on incubating a sample comprising cells of the immune system capable of generating a cell-mediated immune response with at least one antigen at hyperthermic conditions and in the presence of anti-IL-10.

A further aspect of the invention relates to a method to augment a test-antigen specific cell-mediated response based on incubating a sample comprising cells of the immune system capable of generating a cell-mediated immune response with at least one antigen at hyperthermic conditions in the presence of IL-7 and anti-IL-10.

The test-antigen specific cell-mediated immune response can be determined by measuring the level of immune signalling molecules such as IP-10 and/or IFN-γ in response to antigenic stimulation. IP-10 and IFN-γ levels may be detected using ligands such as antibodies specific for IP-10 and IFN-γ or by measuring the level of expression of genes encoding IP-10 and IFN-γ. The present inventors have demonstrated the principle of augmentation of a test-antigen specific cell-mediated response using a method based on M. Tuberculosis specific and BCG-vaccine specific stimulation and subsequent determination of IP-10 and IFN-γ levels. The method can identify persons infected with M. Tuberculosis. The method can also identify persons who have been successfully vaccinated.

The inventors show that incubation at hyperthermic conditions can increase immune signalling molecule responses (IP-10 and IFN-γ) from T-cells and monocytes to antigen and mitogen stimulation (Example 2-4). This effect can be augmented further by addition of the T-cell survival cytokine IL-7 with or without neutralizing antibodies against the anti-inflammatory cytokine IL-10 (anti-IL-10). The inventors have shown that hyperthermic incubation in the presence of anti-IL-10 and IL-7 can increase biomarker production synergistically (Examples 3-4). Thus, the described method leads to higher levels (or higher magnitude) of the immune signalling molecules IP-10 and IFN-γ, compared to traditional methods with incubation at 37° Celsius.

The inventors show that incubation at hyperthermic conditions converted two BCG-vaccinated non-responders, who should theoretically respond, to responders by both IP-10 and IFN-γ. It also converted indeterminate results from two TB patients to positive and negative results respectively. Thus the method reduces the number of false negative and indeterminate test results. Although incubation at hyperthermic conditions increased the background IP-10 production slightly, it reduced background production of IFN-γ (Examples 3-4 and 6). Thus, the method of the present invention is as specific as and more sensitive than tests based on the classic methods using 37° Celsius incubation, and it improves testing and diagnosing of low responders e.g. immunocompromised individuals.

Although IFN-γ is produced mainly by T-lymphocytes and IP-10 is produced mainly by antigen presenting cells such as monocytes, the inventors were not able to show any influence of lymphocyte or monocyte count on biomarker levels (data not shown).

The method described in the present invention solves a series of problems. The currently available methods to monitor cell mediated immunity measures the effect parameter IFN-γ. IFN-γ is expressed at very low levels, close to the limit of even the'most sensitive detection method (in the case of tuberculosis tests, the QuantiFERON test has a cut-off level for positive test at 0.35 international units/ml (17.5 μg/ml) and in the T-SPOT.TB test 5 spot forming units/field). Decreasing cut-off to enhance sensitivity will eventually result in impaired specificity of the tests. Publications based on repeated QuantiFERON tests of people with IFN-γ levels in the lower range have found that IFN-γ levels in this area tend to wobble around the cutoff. I.e. traditional methods to monitor cell mediated immunity are compromised by assay restraints i.e. poor reproducibility at low concentrations of the immune signalling molecule or biomarker. This underlines the potential risk of false positive and false negative results.

The described method increases the responses e.g. the levels of IFN-γ and IP-10 and thereby increases the sensitivity of the cell mediated immune assay e.g. tuberculosis test, and it reduces the risk of false positive and false negative results compared to the traditional test performed at 37° Celsius.

Furthermore patients/donors with low immune responses under normal incubation conditions can be brought to respond by increasing the incubation temperature, especially in the presence of IL-7 and anti-IL-10. Thereby this invention reduces the number patients with false negative test or indeterminate results as it allows for the diagnosis of patients with otherwise inadequate immune responses to antigens.

Thus, this invention improves sensitivity and cost-effectiveness of immuno-diagnostic tests, importantly also in immuno-compromised individuals. Furthermore, it may play a role in vaccine development for both infectious agents and cancer.

The Method

The present invention provides a method for augmentation of a test-antigen specific cell-mediated immune response.

One aspect of the invention relates to the use of an increased temperature during 5 incubation in-vitro of a biological sample with at least one antigen. The incubation at increased temperature leads to an increase in a test-antigen specific cell-mediated immune response.

Thus the present invention relates to a method for generating a test-antigen specific cell-mediated immune response comprising the steps of;
a) providing a sample comprising cells of the immune system capable of generating a cell-mediated immune response from a mammal
b) incubating said sample at hyperthermic conditions with at least one test-antigen
c) determining the test-antigen specific cell-mediated immune response in said sample,
wherein said incubation at hyperthermic conditions generates an augmentation of the test-antigen specific immune response when compared to a reference level obtained by incubation under normal thermal conditions of 37° C.

The test-antigen specific cell-mediated immune response should be understood as a response to an antigen that is specific for the disease or condition one wishes to diagnose. In other words the specificity of the cell-mediated immune response derives from the specificity of the test-antigen.

In the case of vaccines efficacy monitoring, the test-antigen specific cell mediated immune response is generated against an antigen comprised in the vaccine.

An example of a test-antigen specific cell-immune response is the response to ESAT-6 in M. tuberculosis infected individuals.

In a preferred embodiment the sample comprises cells of the immune system capable of generating a cell-mediated immune response. In a particular preferred embodiment the sample comprises immunocompetent cells. Immunocompetent cells are able to produce an immune response such as a cell-mediated immune response after exposure to an antigen.

The Immune Signalling Molecules

The test-antigen specific immune response can be determined by measuring immune signalling molecule production by cells of the immune system in response to specific antigen stimulation.

Thus an aspect of the present invention is a method wherein the test-antigen specific cell-mediated immune response is determined by measuring the level of at least one immune signalling molecule.

Thus according to the present invention the level of 1, 2, 3, 4, 5, 6, 7 or 8 immune signalling molecules are determined. Preferable the level(s) of 1 or 2 or 3 or 4 immune signalling molecules are determined. Most preferable the levels of 1 or 2 immune signalling molecules are determined.

Thus in an preferred embodiment the level of at least 1, at least 2, at least 3, at 15 least 4 or at least 5 immune signalling molecules are measured. Most preferable is the level of at least 1 immune signalling molecule measured.

Most preferable are the levels of 1-2 immune signalling molecules measured, or the level of 1-3 immune signalling molecules or the level of 1-4 immune signalling molecules. In a most preferred embodiment are the levels of 1 or 2 immune signalling molecules measured.

In an embodiment of the present invention measuring the level of more than one immune signalling molecule may reduce the number of false positive and increase the discriminatory power (e.g. increased sensitivity and/or specificity) when applying this method in a diagnostic test. This is useful for instance when using the method as a test that can determine the test-antigen specific cell-mediated immune response in a subject and, in turn, provides means for the diagnosis of e.g. infectious diseases, cancer and/or for monitoring vaccine efficacy.

Thus, in one embodiment, the method further comprises, a step wherein the test-antigen specific cell-mediated immune response is determined by measuring the level of at least one immune signalling molecule.

Thus, in one embodiment, the method further comprises, a step wherein the test-antigen specific cell-mediated immune response is determined by measuring the level of at least one biomarker such as a cytokine or chemokine response.

The immune signalling molecules should be understood as a large family of substances that are either secreted by specific cells of the immune system and/or have an effect on cells of the immune system. Thus, immune signalling molecules are involved in transmitting information between cells such as cells of the immune system.

In a presently preferred embodiment the immune signalling molecule or the at least one signalling molecule is selected from the group of cytokines, chemokines, soluble receptors and soluble receptor antagonists.

In a particular preferred embodiment of the invention the immune signalling molecule is a cytokine or chemokine.

In the most preferred embodiment of the invention the immune signalling molecule is a cytokine.

Cytokines are to be understood as any of a number of substances that are secreted by specific cells of the immune system that carry signals locally between cells, and thus have an effect on other cells. Cytokines can be categorized as signalling molecules. They are proteins, peptides, or glycoproteins. The term cytokine encompasses a large and diverse family of polypeptide regulators that are produced widely throughout the body by cells of diverse embryological origin. Basically, the term “cytokine” refers to immunomodulating agents such as but not limited to interleukins, interferons, etc. The immune signalling molecules is selected from the group consisting of the cytokines INF-γ, IL-2, TNF-α, IL-1b and IL-12.

In another most preferred embodiment of the invention the immune signalling molecule is an interferon.

In another most preferred embodiment of the invention the immune signalling molecule is IFN-γ.

IFN-γ

Interferon-gamma (IFN-γ) is a cytokine that is critical for the immune response against viral and bacterial infections. In humans, the IFN-γ protein is encoded by the IFNG gene. IFN-γ has both immunostimulatory and immunomodulatory effects. IFN-γ is produced predominantly by natural killer and natural killer T cells as part of the innate immune response, and by CD4 and CD8 cytotoxic T lymphocyte effector T cells once antigen-specific immunity develops.

In another most preferred embodiment the immune signalling molecule is a chemokine. Chemokines are a family of small cytokines, or proteins secreted by cells. Their name is derived from their ability to induce directed chemotaxis in nearby responsive cells; they are chemotactic cytokines. These proteins exert their biological effects by interacting with G protein-linked transmembrane receptors called chemokine receptors that are selectively found on the surfaces of their target cells. Chemokines play fundamental roles in the development, homeostasis, and function of the immune system, and they have effects on cells of the central nervous system as well as on endothelial cells involved in angiogenesis or angiostasis. Chemokines are divided into 2 major subfamilies, CXC and CC, based on the arrangement of the first 2 of the 4 conserved cysteine residues; the 2 cysteines are separated by a single amino acid in CXC chemokines and are adjacent in CC chemokines. CXC chemokines are further subdivided into ELR and non-ELR types based on the presence or absence of a glu-leu-arg sequence adjacent and N terminal to the CXC motif. ELR types are chemotactic for neutrophils, while non-ELR types are chemotactic for lymphocytes.

In a preferred embodiment the immune signalling molecules are selected from the group consisting of CC-chemokines.

In another preferred embodiment the immune signalling molecules are selected from the group consisting of CXC-chemokines.

In yet another preferred embodiment the immune signalling molecules are selected from the group consisting of IP-10, MIG, MCP-1, MCP-2, MCP-3.

Other immune signalling molecules relevant for this invention are IL-1RA an antagonist of the IL-1 receptor and sIL-2R a soluble receptor.

In a preferred embodiment of the invention the at least one immune signalling molecule is selected from the group consisting of IP-10, INF-γ, MIG, IL-2, TNF-α, MIP-1a, MCP-1, MCP-2, MCP-3, IL-1b, IL-1RA, sIL-2R, CD40-ligand and IL-12. In another preferred embodiment of the invention at least one immune signalling molecule is selected from the group consisting of IP-10, INF-γ and IL-2.

In another preferred embodiment of the invention the immune signalling molecule is IFN-γ.

In a most preferred embodiment of the invention the immune signalling molecule is IP-10.

The concept of level of immune signalling molecules also covers mathematical manipulations of concentration measurements such as but not limited to multiplication, division and/or addition of at least two cytokine responses.

IP-10

IFN-γ-inducible protein 10 (IP-10) or CXCL10 is a chemokine. The IP-10 gene is mapped to 4q21 by in situ hybridization. IP-10 expression is up regulated by Interferons (IFNs i.e. Interferon gamma (IFN-γ)) and inflammatory stimuli, and it is expressed in many Th1-type inflammatory diseases in a variety of tissues and cell types.

The human gene sequence can be found under ACCESSION number BC010954 (gi 15012099) in Gene Bank.

IP-10 inhibits bone marrow colony formation, has antitumor activity in vivo, is a chemoattractant for human monocytes and T cells, and promotes T cell adhesion to endothelial cells. IP-10 is a potent inhibitor of angiogenesis in vivo. IP-10 may participate in the regulation of angiogenesis during inflammation and tumorigenesis. IP-10 is also a RAS target gene and is overexpressed in the majority of colorectal cancers. Using nuclear magnetic resonance spectroscopy it has been shown that IP-10 interacts with the N terminus of CXCR3 via a hydrophobic cleft formed by the N-loop and 40s-loop region of IP-10, similar to the interaction surface of other chemokines, such as IL8. An additional region of interaction has been identified consisting of a hydrophobic cleft formed by the N terminus and the 30s loop of IP-10. This suggests that a mechanism involving the 30s loop and the configuration of beta strand 2 may account for the interaction and antagonistic function of IP-10 with CCR3.

In the case of tuberculosis high levels of IP-10 have been found in lymph node and lung tuberculous granulomas, in pleural effusions and in the serum or plasma of TB patients as well as in TB-HIV co-infected patients experiencing immune reconstitution syndrome.

Thus, in one embodiment, the method further comprises measuring the level of both IP-10 and IFN-γ. Thus, one aspect of the present invention relates to a method for generating a test-antigen specific cell-mediated immune response comprising the steps of;
a) providing a sample comprising cells of the immune system capable of generating a cell-mediated immune response from a mammal
b) incubating said sample at hyperthermic conditions with at least one test-antigen
c) determining the level of IP10 and/or IFN-γ in said sample, wherein said incubation at hyperthermic conditions generates an augmentation of the test-antigen specific immune response when compared to a reference level obtained by incubation under normal thermic conditions of 37° C.

In yet another embodiment the method further comprises measuring the level of IP-10 and/or IFN-γ and at least one other signalling molecule.

Determination of the level of the signalling molecule(s)

The immune signalling molecules may be detected using ligands such as antibodies specific for the immune signalling molecules or by measuring the level of expression of genes encoding the immune signalling molecules.

Thus in one embodiment the method further comprises a step wherein the immune signalling molecule level is determined by measuring the level of mRNA and/or protein.

In yet another embodiment is the location of the immune signalling molecules in situ determined by methods such as immunofluorescence and microscopy.

Thus, one aspect of the present invention relates to a method for generating a test-antigen specific cell-mediated immune response comprising the steps of;
a) providing a sample comprising cells of the immune system capable of generating a cell-mediated immune response from a mammal
b) incubating said sample at hyperthermic conditions with at least one test-antigen
c) determining the test-antigen specific cell-mediated immune response in said sample,

wherein said incubation at hyperthermic conditions generates an augmentation of the test-antigen specific immune response when compared to a reference level obtained by incubation under normal thermic conditions of 37° C., and wherein said immune signalling molecule level is determined by measuring the level of mRNA and/or protein.

The immune signalling molecule is preferably a cytokine or chemokine such as but not limited to IP-10 and/or IFN-γ. The presence or level of the immune signalling molecule may be determined at the level of the molecule itself or by the extent to which a gene is expressed. The level of immune signalling molecules such as IP-10 and/or IFN-γ is measured by conventional analytical methods, such as immunological methods known to the art.

Measurements of the immune signalling molecule can be combined with measurements of other immune signalling molecules at gene, RNA, or protein level in accordance with the teachings herein.

It is to be understood that any one of the methods described in the present invention is platform independent. Accordingly, any immunological method such as but not limited to ELISA, ELISPOT, Luminex, Multiplex, Immunoblotting, immunochromatographic lateral flow assays, Enzyme Multiplied Immunoassay Techniques, RAST test, Radioimmunoassays, immunofluorescence and various immunological dry stick assays (e.g. lateral flow or cromatographic stick test) may be applicable to the present invention.

As stated above, detection of the immune signalling molecules may be made at the protein or nucleic acid levels. Consequently, reference to presence or level of said immune signalling molecule includes direct and indirect data. For example, high levels of IP-10 mRNA are indirect data showing increased levels of IP-10.

It should be further understood that any method for measuring levels of DNA, RNA and/or mRNA e.g. PCR techniques may be used for measuring the level of the signalling molecules. Methods for measuring the level of signalling molecules at DNA or RNA level are such as but not limited to quantitative PCR (q-PCR), real time PCR (qRT-PCR) and reverse transcription PCR (RT-PCR).

Thus one aspect of the invention relates to a method wherein the determination of the immune signalling molecule level is performed using a method selected from the group consisting of qPCR, RT-PCR, qRT-PCR, ELISA, ELISPOT, Luminex, Multiplex, Immunoblotting, immunochromatographic lateral flow assays, Enzyme Multiplied Immunoassay Techniques, RAST test, Radioimmunoassays, immunofluorescence and various immunological dry stick assays.

Ligands to the immune signalling molecules are particularly useful in detecting and/or quantifying these molecules.

Antibodies to the immune signalling molecules are particularly useful. Techniques for the methods contemplated herein are known in the art and include, for example, sandwich assays, xMAP multiplexing, Luminex, ELISA and ELISpot. Reference to antibodies includes parts of antibodies, mammalianized (e.g. humanized) antibodies, recombinant or synthetic antibodies and hybrid and single chain antibodies.

Both polyclonal and monoclonal antibodies are obtainable by immunization with the immune signalling molecules or antigenic fragments thereof and either type is utilizable for immunoassays. The methods of obtaining both types of sera are well known in the art.

Polyclonal sera are less preferred but are relatively easily prepared by injection of 25 a suitable laboratory animal with an effective amount of the immune signalling molecule, or antigenic part thereof, collecting serum or plasma from the animal and isolating specific sera by any of the known immuno-adsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Detection can also be obtained by either direct measure of a signalling molecule e.g. IP-10 specific antibody in a competitive fluorescent polarization immunoassay (CFIPA) or by detection of homodimerization of interferon-gamma by dimerization induced fluorescence polarization (DIFP). In either case, detection and quantitation will be down to or less than 6 pg/ml.

Several techniques are known to the skilled addressee for determination of biological markers such as IP-10. The presence or level of immune effecter may be determined by ELISA, Luminex, ELISPOT, mRNA based techniques like RT-PCR or Intracellular flow cytometri.

Luminex

Interferon gamma (IFN-γ) has been the gold standard for measuring a Th1 response in infectious disease immunology and especially in TB immunology. IFN-γ determined by Luminex is a poor marker because of lower sensitivity compared to more sensitive methods such as the commercial ELISA developed for the QuantiFERON test.

xMAP or Luminex allows multiplexing of analytes in solution with flow cytometry.

Using a propriety technique, Luminex internally colour codes xMAP microspheres by combining different ratios of two fluorescent dyes. Each bead set is conjugated with a different capture antibody. The use of R-phycoerythrin-labelled detection antibodies allows quantification of antigen-antibody reactions occurring on the microsphere surface, by measurement of the relative fluorescence intensity.

ELISA

Enzyme-linked immunosorbent assay, also called ELISA, enzyme immunoassay or EIA, is a biochemical technique used mainly in immunology to detect the presence of an antibody or an antigen in a sample. In simple terms, in ELISA, an unknown amount of antigen is affixed to a surface, and then a specific antibody is washed over the surface so that it can bind to the antigen. This antibody is linked to an enzyme, and in the final step a substance is added that the enzyme can convert to some detectable signal. In the case of fluorescence ELISA, when light of the appropriate wavelength is shone upon the sample, any antigen/antibody complexes will fluoresce so that the amount of antigen in the sample can be inferred through the magnitude of the fluorescence. Using ELISA to determine the level of at least one immune signalling molecule is described further in the Examples.

Immunochromatographic Tests (ICT)

The principle of ICT (e.g. a lateral flow stick) is an in-vitro immunodiagnostic test that utilizes a primary antibody (Ab) and one to four secondary Ab's all specific for an immune signalling molecule such as IP-10. The primary Ab is attached to colloidal gold and impregnated into a sample pad with a lane containing the secondary Ab in a fixed line.

In the first step the incubated sample is added to the left part of the sample pad. Serum or plasma will flow forward into the lane allowing any IP-10 present to bind to the colloidal gold-labeled primary Ab. The secondary Ab is immobilized in a line across the membrane of the lane. The sample and the labelled primary Ab then migrate along the membrane lane crossing the immobilized secondary Ab line. Test interpretation: Any IP-10 complexed with the gold-labeled primary Ab is captured by the secondary Ab on the membrane and a colour change occurs in the line. The test is then interpreted either a. on the basis of the colour intensity or b. by comparing two tests, one performed on the response sample (e.g. plasma of antigen stimulated test material like whole blood) and one performed on the nil sample, one subtracts the intensity of the colour change in the nil test from the intensity of the colour change in the Ag test and compare this is to a reference.

The readout of the test may also be automated or semi-automated using a computerized interface. This setup could be constructed so the automated interface determines an intensity of the colour change of the line.

In a preferred embodiment the readout is done using a scanner e.g. a flat bed scanner, a reader or a handheld reader. The intensity of the line can be quantified by comparing to a reference e.g. using relevant software.

In another preferred embodiment the readout is done using a camera e.g. a digital camera in a mobile phone. The intensity of the line can be quantified by comparing to a reference e.g. using the naked eye or relevant software. In the case of using a mobile phone camera, the picture can be sent from the phone to a central server for analysis e.g. in an multimedia message service message (or MMS).

In another preferred embodiment the readout an analysis is done using a digital camera in a mobile phone using onboard software.

Immune Modulator

The present invention relates to a method to increase the pro-inflammatory immune response by increasing the incubation temperature of a sample with at least one test antigen in-vitro. Thus, the invention provides a test where incubating cells of the immune system capable of generating a cell-mediated immune response at hyperthermic conditions that potentiates a biomarker response.

As hyperthermia potentially leads to cell stress, a further aspect of the present invention is to add immune modulators to counteract these mechanisms. Thus a particular preferred embodiment of the invention is a method further comprising the step of adding at least one immune modulator to improve the test-antigen specific cell-mediated immune response.

The present invention also relates to improving immunodiagnostic tests by addition of at least one immune modulator.

The term immune modulator is to be understood as a substance that alters the immune response. Immune modulators are substances that are able to induce adjustment of the immune response to a desired level, as in immunopotentiation, immunosuppression, or induction of immunological tolerance and/or are able to counteract potential harmful effects as cell stress due to hyperthermia. An immune modulator is also understood as a substance that is able to boost or inhibit specific areas of the immune system e.g. the T Lymphocytes cells or T lymphocyte subpopulations.

Preferred immune modulators according to the present invention are cytokines and neutralizing antibodies which improve the test-antigen specific cell-mediated immune response. Particular preferred immune modulators are cytokines such as IL-7, IL-15 and IL-21. Other particular preferred immune modulators are neutralizing antibodies binding IL-10, IL-4, and/or IL-5.

One aspect of the present invention is a method further comprising addition of at least one immune modulator wherein the at least one immune-modulator selected from the group consisting of the cytokines IL-7, IL-15, IL-21, neutralizing antibodies binding IL-10, IL-4, IL-5, beads coated with anti-CD25 antibodies, beads coated with anti-CD39 antibodies, sense or antisense oligonucleotide to genetic material encoding IL-10, JAKI or TYK2, a CpG containing oligonucleotide, an oligonucleotide acting as a TLR modulating agent, and a TLR modulating agent is added in step b. The effects of addition of immune modulators is exemplified by antibodies towards IL-10 and addition of IL-7 in the examples.

In a preferred embodiment the at least one immune modulator is a cytokine selected from the group consisting of IL-7, IL-15 and IL-21.

In another preferred embodiment the at least one immun modulator is a neutralizing antibody selected from the group consisting of the neutralizing antibodies binding IL-10, neutralizing antibodies binding IL-4, neutralizing antibodies binding IL-5 and neutralizing antibodies binding CD25.

In yet another preferred embodiment the at least one immune modulator is selected from the group consisting of beads coated with anti-CD25 antibodies, sense or antisense oligonucleotide to genetic material encoding IL-10, JAKI or TYK2, a CpG containing oligonucleotide, an oligonucleotide acting as a TLR modulating agent, and a TLR modulating agent.

It is well established that the cytokine Interleukin-7 (IL-7) is essential for survival and homeostasis of naïve and memory CD4+ and CD8+ T-cell subsets. Hyperthermia might cause cell stress and damage and addition of the survival cytokine IL-7 during incubation may protect from these potentially harmful effects.

A particular preferred embodiment of the present invention relates to a method where IL-7 is added to protect the cells from the potentially harmful effects of incubation at hyperthermic conditions.

Another aspect of the present invention is addition of antibodies against the anti-inflammatory cytokine IL-10 to further boost the pro-inflammatory responses.

It is shown by the present invention that incubation at hyperthermic conditions or adding IL-7 with or without anti-IL-10 increases the test-antigen specific cell-mediated immune response.

A preferred embodiment of the present invention relates to a method where IL-7 is added to protect the cells from these potentially harmful effects of hyperthemal incubation with or without addition of antibodies against the anti-inflammatory cytokine IL-10 in order to further boost the pro-inflammatory responses.

Thus the present invention also relates to a method for improving immunodiagnostic tests by addition of IL-7 with or without neutralizing antibodies binding IL-10.

The present invention also concerns incubation at hyperthermic conditions in the presence of IL7 with or without anti-IL10. The presence of both IL-7 and anti-IL-10 appears to provide optimal incubation conditions for production of immune signalling molecules both antigen dependent and mitogen induced.

The invention also relates to a test system that can detect infection with e.g. M Tuberculosis based on measuring immune signalling molecules e.g. the chemokine IP-10 and/or cytokine IFN-γ following stimulation of cells of the immune system capable of generating a cell-mediated immune response with antigenic proteins/peptides at hyperthermic conditions and/or in the presence of IL-7 and/or anti-IL-10.

The described test system is more sensitive than tests performed at normal incubation temperatures i.e. 37° C., and it reduces the number of false negative and indeterminate test result. It improves testing and diagnosing. According to the present invention patients/donors with low immune responses under normal incubation conditions can be brought to respond by increasing the incubation temperature, especially in the presence of IL-7 and anti-IL-10. Thereby this invention allows forthe diagnosis of patient with otherwise inadequate immune responses to antigens. Furthermore this invention improves sensitivity and cost-effectiveness of immuno-diagnostic tests, importantly also in immuno-compromised individuals. Furthermore, it may play a role in vaccine development for infectious agents and cancer.

Il-7

Interleukin 7 (IL-7) is a protein that in humans is encoded by the IL7 gene. IL-7 is a survival cytokine. IL-7 is known to stimulate proliferation of lymphoid progenitor cells and is important for B and T cell development. Cytokine Interleukin-7 has been shown to be essential for survival and homeostasis of naïve and memory CD4+ and CD8+ T-cell and defect IL-7 signalling results in severe immunodeficiency in humans.

IL-10

Interleukin-10 (IL-10), also known as human cytokine synthesis inhibitory factor (CSIF), is an anti-inflammatory cytokine. In humans IL-10 is encoded by the IL10 gene. IL-10 is known to be an important immune regulatory molecule. It is capable of inhibiting synthesis of pro-inflammatory cytokines like IFN-γ, IL-2, IL-3, TNFα and GM-CSF from cells such as macrophages and Th1 cells. IL-10 is also a potent suppressor of the antigen presenting capacity of antigen presenting cells. However, it is also stimulatory towards certain T cells and mast cells and it stimulates B cell maturation and antibody production. Neutralizing antibodies have been developed that specifically inhibit IL-10 thereby inhibiting or neutralizing the biological effects of IL-10. Silencing oligonucleotides binding DNA and/or RNA are also effective in inhibiting IL-10 mediated signals.

Immunomodulation of Cell Populations

In another embodiment the assay can be further potentiated by inhibiting anti-inflammation. This can be done by inhibition, depletion or elimination of cell populations that inhibit the test-antigen cell mediated immune response such as regulatory T-cells or Th2 cells.

Preferred immune modulators inhibit the function or activity of T-regulatory cells. These immuno modulator are selected from the group consisting of CD25 ligands; sense or antisense oligonucleotides to genetic material encoding janus kinase 1 (JAKI) or Thyrosine kinase 2 (TYK2); a CpG containing oligonucleotides; oligonucleotides acting as TLR modulating agents; and a TLR modulating agents. More specifically immune modulators with T regulatory cell inhibiting qualities are anti-CD25 antibodies, and/or phosphorothioated oligonucleotides. More specifically an oligonucleotide can be complementary or homologous to genetic material (RNA or DNA) encoding a JAKI or TYK2 molecule to augment or enhance the sensitivity of an immune cell-mediated assay. The oligonucleotides contemplated herein may have a modified backbone or have chemically modified nucleotides or nucleosides such as phosphorothioates-modified oligonucleotide.

One aspect of the present invention is a method further comprising addition of at least one immune modulator wherein the at least one immune-modulator is anti-CD25 antibodies coated on the surface of a bead.

Test Antigen

The present invention relates to a method to augment a test-antigen specific cell-mediated immune response. The choice of antigen suitable for the present invention, also referred to as test-antigen(s), is any antigen where it is desired determines the effect of said antigen on the cell-mediated immune response.

Test-antigen should be understood as an antigen that is specific for the disease or condition one wishes to diagnose.

Test-antigens may be in the form of peptide, polypeptide or protein, carbohydrate, glycoprotein, phospholipid, phosphoprotein or phospholipoprotein or non-protein chemical agent.

An example of a test-antigen is ESAT-6, an antigen which is almost exclusively expressed in M. tuberculosis and can be considered specific for M. tuberculosis. ESAT-6 peptides are presented on antigen-presenting cells and are recognized by T cells carrying a T cell receptor specific for the ESAT-6 antigen.

An example of an unspecific antigen is purified protein derivate (PPD) of Bacille Calmette et Guérin (BCG) or tuberculin PPD of M. tuberculosis, M. bovis or M. avium. PPD is a protein precipitate comprising several non-specific antigens. It is well established that PPD cross reacts with most mycobateria species (including M. Leprae that causes the disease leprosy) and furthermore PPD has unspecific effects which include mitogen-like effects. Hence PPD cannot be considered as a test-antigen—i.e. PPD is an unspecific antigen.

Other examples of unspecific antigens are antigens that elicit a strong innate immune response e.g. lipopolyshaccaride (LPS) which is recognized by both T cells and innate receptors. Although LPS comprise specific antigens that could be classified as test-antigens, the innate responses are often stronger and obscures the test-antigen induced signals. As the innate responses are independent of immunological memory they are incapable of generating specific signals that can differentiate whether or not the mammal has previously encountered the specific test-antigen and thus generated immunological reactivity to the specific test-antigen(s) or previously encountered other antigens generating immunological cross reactivity to the specific test-antigen(s),

In other words the specificity of the cell-mediated immune response derives from the specificity of the test-antigen.

Depending on the degree of specificity needed for evaluating of the disease or condition, the skilled addressee can select test-antigens of varying specificies.

In one aspect of the invention the choice of test-antigen suitable for the present invention depends on the type of infection that the skilled addressee would like to assess. Accordingly the selected antigens are disease associated. For example when monitoring M. Tuberculosis infection any available M. Tuberculosis antigens could generate the necessary response and vice versa. Several test-antigens are already used in the existing commercial assays.

Wherein the infection is believed to be related to tuberculosis, the antigen or the at least one antigen is a RD-1 and/or RD-11 antigen.

In a preferred embodiments is the antigen selected from the group consisting of RD-1 antigens, ESAT-6, CFP-10, TB7.7, the fusion protein ESAT-6/CFP-10, TB10.4 and fusion proteins combining several different but specific antigens.

An example of a test-antigen specific biomarker response is the stimulation with the M. tuberculosis specific antigens ESAT-6, CFP10 and TB7.7 exemplified in e.g. example 6)

In another preferred embodiment is the antigen or at least one antigen selected from the group consisting of the following latency antigens from the in vivo expressed genes: Rv0079, Rv0570, Rv0717, Rv1170, Rv1284, Rv1363, Rv1956, Rv2034, Rv2225, Rv2324, Rv2380, Rv2435, Rv2465, Rv2737c, Rv2838c, Rv2982c, Rv3353c, Rv3420c and Rv3515.

In yet another preferred embodiment is the antigen or at least one antigen selected from the group consisting of the following antigens from the Enduring Hypoxic Response genes: Rv0140, Rv0244c, Rv0251, Rv0384c, Rv0753c, Rv0767, Rv0846, Rv0847, Rv0967, Rv0990, Rv0991, Rv1284, Rv1403, Rv1471, Rv1733, Rv1806, Rv1874, Rv1875, Rv1909, Rv1955, Rv1956, Rv1957, Rv2034, Rv2035, Rv2324, Rv2389, Rv2465, Rv2466, Rv2558, Rv2626, Rv2627, Rv2628, Rv2642, Rv2643, Rv2658, Rv2660, Rv2662, Rv2745, Rv2913, Rv3223, Rv3406, Rv3515, Rv3536 and Rv3862.

Test-antigens relevant for the present invention can be modified e.g. by coupling with invariant chain from MHC to improve immunogenicity without compromising specificity.

Preferred antigens are also the NTM sensitins selected from the list consisting of M. avium, M. gordonae and M. xenopi.

In a presently preferred embodiment the test-antigen or the at least one test-antigen is selected from the group consisting of ESAT-6, CFP-10, TB7.7, and other RD-1 and RD-11 antigens.

In yet an embodiment of the present invention the test-antigen is ESAT-6.

In another embodiment of the present invention the test-antigen is CFP-10.

In a further embodiment of the present invention the test-antigen is TB 7.7.

In a presently preferred embodiment of the present invention the test-antigens are RD antigens.

In a presently preferred embodiment of the present invention the test-antigens are RD-1 antigens.

In yet a presently preferred embodiment of the present invention the antigens are RD-11 antigens.

Several research institutions are working on identification of test-antigens solemnly expressed by the individual infectious agent, so called microbe- or disease-specific antigens. In the case of M. tuberculosis, specific test-antigens are expressed at different stages of infection such as but not limited to dormant, latent, active, recent, pulmonary, extrapulmonary, localized or cured stages.

The present invention can be implemented using such test-antigens thus providing a tool for identification of that specific stage (e.g. latent infection with M. tuberculosis).

In a preferred embodiment, several antigens from the same microorganism can 10 be added when generating the response sample. By adding several antigens with various tissue type preferences the strength of the method is increased. In the case of tuberculosis, combining antigen-peptides of ESAT-6, CFP-10 and TB7.7 proteins ensures that the test covers a broad range of tissue types and thus gives stronger and more reliable test results in different patient populations.

Wherein the infection is believed to be related to Chlamydia, the antigen is selected from the group consisting Serovar D extract, major outer membrane protein (MOMP), cysteine-rich outer membrane proteins (OMPs), OMP2, OMP3, Poly-morphic OMPs (POMPs), adenosine diphosphate/adenosine triphosphate translocase of Chlamydia pneumonia, porin B proteins (PorBs), and CT521.

As apparent from the present invention the source of infection may vary. In an embodiment of the present invention the antigen is selected from the group consisting of fixed-epimastigotes, fixed-trypomastigotes, disrupted-epimastigotes, disrupted-trypomastigotes, purified antigenic fractions from epimastigotes, semipurified antigenic fractions from epimastigotes, trypomastigote excretory-secretory antigens (TESA), predominant variable antigen type (VAT), variable surface glycoprotein (VSG), trans-sialidase (TS) e.g. TS13, amastigote surface protein-2 (ASP2), KMP-11m, CRA, Ag30, 3L8, TCR27, Agl, JL7, H49, TCR39, PEP-2, Ag36, 3L9, MAP, SAPA, TCNA, Ag13, TcD, B12, TcE, JL5, A13, 1F8, Tc-24, Tc-28, Tc-40, Cy-hsp70, MR-HSP70, Grp-hsp78, CEA, CRP, SA85-1.1, FCaBP (flagellar Ca2+-binding protein), FL-160 (flagellar surface protein of 160 kDa) and, FRA (flagellar repetitive antigen) said antigens being related to Trypanosomas.

In a preferred embodiment of the present invention the antigen is selected from the group consisting of fixed-epimastigotes, fixed-trypomastigotes, disrupted-epimastigotes, disrupted-trypomastigotes, purified antigenic fractions from epimastigotes, semipurified antigenic fractions from epimastigotes, trypomastigote excretory-secretory antigens (TESA), predominant variable antigen type (VAT), variable surface glycoprotein (VSG), trans-sialidase (TS) e.g. TS13, amastigote surface protein-2 (ASP2), FCaBP (flagellar Ca2+-binding protein), FL-160 (flagellar surface protein of 160 kDa) and FRA (flagellar repetitive antigen).

In the case wherein the infection is related to schistosoma, the antigen is selected from the group consisting of disrupted schistosoma egg, excreted/secreted glycoproteins (ES), tegumental (TG) glycoproteins, soluble egg antigen (SEA), soluble extract of S. mansoni (SWAP), keyhole limpet haemocyanin (KLH), RP26, Sj 31, Sj 32, paramyosin, Sm62-IrV5, Sm37-SG3PDH, Sm28-GST, Sm14-FABP, PR52-filamin PL45-phosphoglycerate kinase, PN18-cyclophilin, MAP3, Sm23, MAP4, Sm28-TPI, Sm97, CAA, CCA and, Schistosoma mansoni heat shock protein 70.

In a preferred embodiment of the present invention the antigen is selected from the group consisting of excreted/secreted glycoproteins (ES), tegumental (TG) glycoproteins, soluble egg antigen (SEA), soluble extract of S. mansoni (SWAP), keyhole limpet haemocyanin (KLH) and, RP26.

In respect of leishmania, the antigen or at least one antigen is selected from the group consisting of disrupted promastigozyes, leishmanin, rGBP, rORFF, rgp63, rK9, rK26, rK39, PN18-cyclophilin, MAP3, Sm23, MAP4, Sm28-TPI, Sm97, CAA and, CCA.

In fact any test-antigen specific for the species to be analyzed could be useful according to the present invention.

One aspect of the present invention relates to a method wherein the test-antigen specific cell-mediated immune response is used to detect a cancer or neoplasm or malignancy.

In respect to cancer, the test antigen is selected from the group consisting of WT1, MUC1, LMP2, HPV E6, HPV E7, EGFRvIII, Her2, neu, MAGE A3, p53 mutant and non mutant, NY-EOS-1, PSMA, GD2, CEA, MelanA, MART1, Ras-mutant, gp100, PR1, Bcr-abl, Thyrosinase, surviving, PSA, hTERT, sarcoma translocation breakpoints, EphA2, PAP, ML-IAP, AFP, EpCAM, ERG, NA17, PAX3, ALK, Androgen receptor, Cyclin B1, Polysialic Acid, NYCN, TRP-2, RhoC, GD3, Fucosyl GM1, mesothelin, PSCA, MAGE A1, sLe(a), CYP181, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, Sperm fibrous shealth proteins, AKAP-4, SSX2, XAGE 1, B7H3, Legumain, Tie2, Page4, VEGFR2, MAD-CT-2 and FAP.

In fact any test-antigen specific for cancer, precancer or mutation could be useful to diagnose and monitor cancer according to the present invention.

In another preferred embodiment, a range of different antigens from different diseases can be combined to enable a screening tool with low specificity for the individual disease, but high sensitivity for “infection”. A kit combining e.g. a palette of antigens from microbes soldiers are exposed to during mission (e.g. malaria, tuberculosis, leishmania, schistosoma and/or trypanosomiasis) will enable doctors to perform one quick screening-test instead of a range of different tests.

In another preferred embodiment, a range of different antigens from different sexually transmitted diseases can be combined to enable a screening tool with low specificity for the individual disease, but high sensitivity for sexually transmitted diseases (STD). A kit combining e.g. a palette of antigens from microbes which transmit infection with intimate mucosal contact (e.g. Haemophilus ducreyi, Chlamydia trachomatis, Klebsiella granulomatis, Neisseria gonorrhoeae, Treponema pallidum, Trichophyton rubrum, Candidiasis, herpes, Hepatitis B virus, HSV, HIV, HPV, MCV, Phthirius pubis, Sarcoptes scabiei) will enable doctors to perform one screening-test instead of a range of different tests. Such a test would serve as a quick screening tool for STD in risk seeking individuals such as but not limited to sex workers.

In another preferred embodiment, combined kits may comprise antigens from various microbes infecting an organ (e.g. Nesseria and Chlamydia species causing pelvic inflammatory disease), or comprise antigens from infectious agents that cause common symptoms (e.g. treatable diarrhoea caused by campylobacter and shigella infection could be distinguished from untreatable diarrhoea caused by virus e.g. rotavirus).

Sample

One embodiment of the present invention contemplates a method for generating a test-antigen specific cell-mediated immune response, said method comprising providing a sample comprising cells of the immune system capable of generating a cell-mediated immune response from a mammal, incubating said sample at hyperthermic conditions with at least one test-antigen and determining the test-antigen specific cell-mediated immune response in said sample.

Another embodiment of the present invention contemplates a method for generating a test-antigen specific mediated immune response in a subject, said method comprising collecting a sample from said subject wherein said sample comprises cells of the immune system, which are capable of producing immune signalling molecules following stimulation by at least one test-antigen, incubating said sample at hyperthermic temperature with said at least one test-antigen and then measuring the presence of or elevation in the level of at least one immune signalling molecule wherein the presence or level of said immune signalling molecule is indicative of the capacity of said subject to mount a test-antigen specific cell-mediated immune response.

In one embodiment the sample is derived from whole blood or cells derived from blood, pleural fluid, bronchial fluid, tissue biopsies, ascites liquid, and/or cerebrospinal fluid.

In a preferred embodiment the sample is derived from blood.

In a particular preferred embodiment the sample comprises cells selected from the group consisting of peripheral blood mononuclear cells (PBMC's), T cells, CD4 T cells, CD8 T cells, gamma-delta T cells, monocytes, macrophages, dendritic cells and NK cells.

Conveniently, when the sample is whole blood, the blood collection tube is treated with anticoagulant (e.g. heparin) and optionally an immune modulator and optionally nutrients. Notwithstanding that whole blood is the preferred and most convenient sample, the present invention extends to other samples containing cells of the immune system capable of generating a cell-mediated immune response such as but not limited to pleural fluid, ascites fluid, lymph fluid, spinal or cerebral fluid, tissue fluid and respiratory fluid including nasal, and pulmonary fluid.

Reference to “whole blood” includes whole blood which has not been diluted such as with tissue culture, medium, reagents, excipients, etc. In one embodiment, the term “whole blood” includes an assay sample (i.e. reaction mixture) comprising at least 10% by volume whole blood. The term “at least 10% by volume” includes blood volumes of 10, 11, 12, 13, 14, 15, 16, 17, 18, 19, 20, 21, 22, 23, 24, 25, 10 26, 27, 28, 29, 30, 31, 32, 33, 34, 35, 36, 37, 38, 39, 40, 41, 42, 43, 44, 45s 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100% by volume of total assay volume of the reaction mixture. Additional agents may be added such as culture media, enzymes, immune modulators, excipients antigen and the like without departing from the sample comprising “whole blood”.

In one embodiment the present invention thus relates to a method, wherein the sample is whole blood, or cells derived from blood, pleural fluid, bronchial fluid, tissue biopsies, ascites liquid, and/or cerebrospinal fluid.

In another embodiment the present invention thus relates to a method, wherein the sample comprises cells selected from the group consisting of peripheral mononuclear cells, T cells, CD4 T cells, CD8 T cells, gamma-delta T cells, monocytes, macrophages, dendritic cells and NK cells.

In one embodiment, the sample is whole blood, which may be collected in three suitable containers in which antigen, mitogen or “nil” are present. In another embodiment, antigens, mitogen or “nil” can be added afterwards to aliquots containing the sample e.g. whole blood.

In another embodiment, the sample is whole blood which may be collected in collection tubes containing the antigen, mitogen or “nil” or to aliquots of whole blood to which antigen, mitogen or nil is added.

In a preferred embodiment, the sample is whole blood which is collected into a evacuated tube coated with dried test-antigens optionally with a substance providing nutrients for the cells (e.g. in the form of carbohydrates) and optionally with an immune modulator.

In another preferred embodiment the sample is collected into an approx. 3-4 mm diameter capillary tube.

Generally, blood is maintained in the presence of an anticoagulant (preferably heparin, alternatively e.g. citrate or EDTA). The anticoagulant is present in the blood collection tube when blood is added. The use of blood collection tubes is preferably but not necessarily compatible with standard automated laboratory systems and these are amenable to analysis in large-scale and random access sampling. Blood collection tubes also minimize handling costs and reduce laboratory exposure to whole blood and plasma and, hence, reduce the risk of laboratory personnel contracting a pathogenic agent such as but not limited to human immunodeficiency virus.

Aliquots of whole blood may be in volumes ranging from 10 μL-4000 μl, such as but not limited to 10 μL, 20 μL, 30 μL, 40 μL, 50 μL, 60 μL, 70 μL, 80 μL, 90 μL, 100 μl, 200 μl, 300 μl, 400 μl, 500 μl, 501 μL, 525 μl, 550 μL, 600 μl, 700 μl, 800 μl, 900 μl, 1000 μl, 1100 μl, 1200 μl, 1300 μl, 1400 μl, 1500 μl, 1600 μl, 1700 μl, 1800 μl, 1900 μl, 2000 μl, 2100 μl, 2200 μl, 2300 μl, 2400 μl, 2500 μl, 2600 μl, 2700 μl, 2800 μl, 2900 μl or 3000 μl.

Sample can be incubated in tubes, tissue culture wells or other containers and antigen, mitogen and “nil” can be added in relevant concentrations.

A blood collection-tube includes a vaccutainer-tube or another similar vessel, but blood can also be drawn directly into an open tube or a capillary tube.

Incubation at Hyperthermic Conditions

The cells of the cell-mediated immune system lose the capacity to mount a cell-mediated immune response in whole blood after extended periods following blood draw from the subject. Responses are often severely reduced or absent 24 hours following blood draw, if the blood sample is not treated in a manner that prolongs the life of the cells such as, but not limited to, preservation at a temperature between 10° and 39° C. Celsius.

In one embodiment the reduction of labour allows stimulation of sample with antigens to be performed at point of care locations such as the physicians' offices, clinics, outpatient facilities, veterinary clinics or on farms. Once antigen stimulation is complete, the requirement for fresh and active cells no longer exists. IP-10 and other biomarkers such as cytokines or immune signalling molecules are stable in plasma and, the sample can thus be stored, frozen or shipped without special conditions or rapid time requirements in a similar fashion to standard plasma samples used for other infectious disease or other disease diagnosis.

The incubation step may be from 5 to 144 hours, more preferably 5 to 120 hours and even more preferably 12 to 24 hours or a time period in between. Thus in one embodiment of the present invention the incubation time is 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 26 hours, 30 hours, 36 hours, 42 hours, 48 hours, 72 hours, 96 hours, 120 hours, or 144 hours.

In the present invention the incubation step is performed at hyperthermic conditions. Hyperthermic conditions include incubation at a temperature ranging from 38° to 42° Celsius.

The incubation step can take place at a temperature ranging from 38° to 42° Celsius. Thus, in one embodiment of the present invention the incubation temperature may be 38° Celsius, 38.1° Celsius, 38.2° Celsius, 38.3° Celsius, 38.4° Celsius, 38.5° Celsius, 38.6° Celsius, 38.7° Celsius, 38.8° Celsius, 38.9° Celsius, 39.0° Celsius, 39.1° Celsius, 39.2° Celsius, 39.3° Celsius, 39.4° , Celsius, 39.5° Celsius, 39.6° Celsius, 39.7° Celsius, 39.8° Celsius, 39.9° Celsius, 40.0° Celsius, 40.1° Celsius, 40.2° Celsius, 40.3° Celsius, 40.4° Celsius, 40.5° Celsius, 40.6° Celsius, 40.7° Celsius, 40.8° Celsius, 40.9° Celsius, 41.0° Celsius, 41.2° Celsius, 41.3° Celsius, 41.4° Celsius, 41.5° Celsius, 41.6° Celsius, 41.7° Celsius, 41.8° Celsius, 41.9° Celsius or 42.0° Celsius.

In a more preferred embodiment is the incubation temperature is 38.5° Celsius, 39.0° Celsius, 39.5° Celsius, 40.0° Celsius, or 40.5° Celsius.

The incubating step can be performed at a not fixed temperature between, but not limited to, 38° to 42.0° Celsius, more preferably from 38.0° to 41°, more preferably from 38.2° to 40.7°, more preferably from 38.5° to 40.5° Celsius and even more preferably from 39.0° to 40.0° Celsius.

Thus, a preferred embodiment of the present invention is wherein said hyperthermic conditions are incubation at a temperature between 38.5-41.0° Celsius.

In a more preferred embodiment is wherein said hyperthermic conditions is incubation at a temperature between 39-40° Celsius

Several methods are known to the skilled addressee for incubating the samples at hyperthermic conditions. It is possible to use for instance incubators, water baths and heating blocks.

One embodiment of the invention allows stimulation of sample in dilution to be performed, this with addition of culture media to the cell culture.

Another embodiment of the invention allows stimulation of sample to be performed with addition of inert dilution liquid (e.g. saline) to the cell culture.

It is to be understood that in the present invention it is preferred that at least one carbohydrate is present when incubating the sample with the at least one test-antigen. The presence of carbohydrate improves cell culture growth.

Thus the invention also relates to a method wherein it is preferred that at least one carbohydrate such as a smaller carbohydrate e.g. a monosaccharide and disaccharide are present in said incubation step.

A particular preferred embodiment is a method wherein sugar in the form of hydrocarbons and/or glycans is added in the incubation step.

In a most preferred embodiment is the added sugar dextrose.

This carbohydrate e.g. dextrose may be added in said incubation step.

Augmentation

One aspect of the invention relates to an augmentation of the test-antigen cell-mediated immune response by incubation at hyperthermic temperatures.

The augmentation can be determined by subtracting the level of the at least one signalling molecule after incubation at hyperthermic conditions with a reference-level. As understood by the skilled addressee the reference-level can be determined by incubating said sample comprising cells of the immune system capable of generating a cell-mediated immune response with at least one test-antigen at 37° C.

In a preferred embodiment the augmentation of the test-antigen cell-mediated immune response is to be understood as an improvement of the test-antigen cell-mediated immune response which can be determined by measuring the level of the at least one immune signalling molecule.

This improvement or augmentation is in a preferred embodiment to be understood as a higher level of the at least one immune signalling molecule after incubation of said sample at hyperthermic temperatures than after incubation at 37° C.

This improvement or augmentation is in a preferred embodiment to be understood as a higher magnitude of the at least one immune signalling molecule after incubation of said sample at hyperthermic temperatures than after incubation at 37° C.

This improvement or augmentation is in a preferred embodiment to be understood as a higher number of molecules (measurable e.g. using pg/ml or moles/ml) of the at least one immune signalling molecule after incubation of said sample at hyperthermic temperatures than after incubation at 37° C.

This improvement or augmentation is in a preferred embodiment to be understood as a higher concentration of molecules of the at least one immune signalling molecule after incubation of said sample at hyperthermic temperatures than after incubation at 37° C.

In a particular preferred embodiment the improvement is to be understood as an absolute higher level of the at least one immune signalling molecule after incubation of said sample at hyperthermic temperatures than after incubation at 37° C. The absolute level is to be understood as the level of the at least one immune signalling molecule determined in the sample i.e. without subtracting background levels of the at least one immune signalling molecule.

In another preferred embodiment the improvement is to be understood as a higher level of the at least one immune signalling molecule after incubation of said sample at hyperthermic temperatures than after incubation at 37° C. after subtracting background levels of the at least one immune signalling molecule.

The improvement is in a particular preferred embodiment to be understood as an improved signal to noise ratio of the test-antigen specific cell-mediated immune response.

Thus in a particular preferred embodiment, the present invention relates to a method, wherein said method generates an improved signal to noise ratio of the test-antigen specific cell-mediated immune response. This is exemplified in example 7.

In general the term signal-to-noise ratio compares the level of a desired signal (the test-antigen specific cell-mediated immune response in this case measured by the level of a immune signalling molecule/the level of a immune signalling molecule resulting from the test-antigen specific cell-mediated immune response) to the level of background noise (the level of said immune signalling molecule present in an unstimulated sample). The higher the ratio, the less obtrusive the background “noise” is. The improved signal to noise ratio of the test-antigen specific cell-mediated immune response can be achieved by lowering the background levels of the immune signalling molecule and/or by increasing the level of test-antigen specific cell-mediated immune response.

The background level is determined by measuring the level of immune signalling molecule in an unstimulated sample (identical to the sample which is incubated with the test-antigen).

The signal to noise ratio can be determined by dividing the level of the immune signalling molecule in the stimulated sample with the level of said immune signalling molecule in the unstimulated sample

An improved signal to noise ratio by incubation of hyperthermic temperatures can be determined by determining the signal to noise ratio for a sample and a specific test-antigen or at least one specific test-antigen at both 37° Celsius and at hyperthermic temperatures e.g. 39° Celsius.

An improved signal to noise level is to be understood as when the signal to noise ratio is increased when comparing the signal to noise ratio determined for said test antigen at 37° Celsius and said test antigen at hyperthermic temperatures such as 39° Celsius. Thus the signal to noise ratio achieved at hyperthermic temperatures is better than at 37° Celsius.

Synergistic Effect

Synergy is defined as the advantageous corporation of different entities for a final outcome. I.e. the interplay between two or more entities/or modifications) will produce an overall better result than the sum of each entity alone. In accordance with this invention and as demonstrated in the examples hyperthermic incubation acts in synergy with the added immune modulators, both in pairs and all together.

Reference and Cut-Off Levels

As will be generally understood by those of skill in the art, methods for screening for cell-mediated immune reactivity are processes of decision-making by comparison. For any decision-making process, reference-values based on subjects having the disease or condition of interest and/or subjects not having the disease, infection, or condition of interest are needed, additionally for the present invention such comparison on the various temperature levels in the hyperthermic region as described herein is also necessary.

The cut-off level can be adjusted based on several criteria such as but not restricted to certain groups of individuals tested. E.g. the cut-off level could be set lower in individuals with immunodeficiency and in patients at great risk of progressing to active disease, cut-off may be higher in groups of otherwise healthy individuals with low risk of developing active disease.

The discriminating value is a value which has been determined by measuring the parameter or parameters in both a 37° Celsius control group or samples and a group or samples at the hyperthermic conditions determining the discriminating value(s). The discriminating value can be determined using receiver operation characteristics curves (ROC curves). The discriminating value determined in this manner is valid for the same experimental setup in future individual tests.

Measurements of e.g. biomarker concentration can be translated to international units (IU). IU relates to the biological activity of the biomarker and is a reference to benchmark between various methods of measurements.

In other embodiments of the invention the determined cut-off value can be combined with a stimulation-index defined for example as antigen-stimulated IP-10 concentration divided by the un-stimulated plasma concentration.

Although any of the known analytical methods for measuring the levels of these analytes will function in the present invention, as obvious to one skilled in the art, the analytical method used for each marker must be the same method used to generate the reference data for the particular marker. If a new analytical method is used for a particular marker or combination of markers, a new set of reference data, based on data developed with the method, must be generated.

The multivariate discriminant analysis and other risk assessments can be performed on the commercially available computer program statistical package Statistical Analysis System (manufactured and sold by SAS Institute Inc.) or by other methods of multivariate statistical analysis or other statistical software packages or screening software known to those skilled in the art.

As obvious to one skilled in the art, in any of the embodiments discussed above, changing the risk cut-off level of a positive test or using different a priori risks which may apply to different subgroups in the population, could change the results of the discriminant analysis for each patient.

In the context of the present invention, the term “reference” relates to a standard in relation to quantity, quality or type, against which other values or characteristics can be compared, such as e.g. a standard curve.

Diagnosis

In one embodiment, and as stated previously, the method may be used for diagnosis of subjects suspected of various immunological states, such as infections. When used in diagnosis the method according to the present invention may help to determine the presence of immunological states, such as infections, usually accomplished by evaluating clinical symptoms and further laboratory tests. The test may diagnose various stages of infection i.e. a recently encountered infection in an individual without any symptoms, an infection encountered many years back in an individual with no symptoms of that infection or an active infection where the patients has symptoms due to the infection.

Thus, the invention relates to a method for determination of the potential or capacity of a subject to mount a test-antigen specific cell-mediated immune response. The present invention provides a method that allows for detection of e.g. infection with tuberculosis, based on measuring immune signalling molecules e.g. the chemokine IP-10 and/or cytokine IFN-γ following stimulation of cells of the immune system capable of generating a cell-mediated immune response with antigenic proteins/peptides at hyperthermic conditions and/or in the presence of IL-7 and/or anti-IL-10.

Thus a particular preferred embodiment of the present invention relates to a method wherein said test-antigen specific cell-mediated immune response is used to diagnose an infection caused by a microorganism capable of expressing the said test antigen.

The described test system reduces the number of false negative test results and indeterminate test results and is more sensitive than tests comprising an incubation step at 37° Celsius. Thus, the method according to the present invention improves testing and diagnosing.

In another embodiment the method may be used for diagnosis of subjects suspected of tuberculosis (e.g. active, latent or recent TB infection) and in particular patients at increased risk for progression from latent to active tuberculosis i.e. patients receiving immunosuppressing medication (i.e. monoclonal antibody treatment (anti-CD20 antibodies (e.g. Rituximab©) or TNF-α blocking treatment (e.g. Remicade©, Enbrel©, Humira©))) or steroids or cancer-chemotherapy; or, patients suffering from immunosuppressing conditions (e.g. HIV infection, cancer, IDDM or non-insulin dependent diabetes mellitus (NIDDM), autoimmune conditions, malnutrition, old age, intravenous drug use (IVDU) or inherited immune disorders), and in individuals who have recently been infected. In fact following standard guidelines these patients should be screened for active, latent or recent TB before initiation of medical treatment.

Currently it is strongly recommended to screen patients who are candidates for TNF-α blocker treatment or are HIV positive with either TST or a M. tuberculosis antigen-specific IFN-γ test. However, studies have shown that these tests are unreliable in the above mentioned patient categories and thus this method is a better choice as it reduces the number of false negative test results and indeterminate test results

In another embodiment the method may be used to screen individuals suspected of Chlamydia infection (e.g. uro-genital infection, pelvic infection and/or infection in the eye).

Accordingly the methods of the present invention may be applicable for screening of persons at high risk of infectious diseases e.g. persons who have been staying in or travelling through disease endemic areas.

Thus, in an embodiment of the present invention the infectious diseases are selected from the group consisting of malaria, tuberculosis, meningitis, Japanese encephalitis, cholera, leishmanina, dengue and polio.

In another embodiment of the invention the infectious diseases are selected from the sexually transmitted diseases consisting of chancroid, Chlamydia infection, Gonorrhea, Lymphogranuloma venereum, Ureaplasma urealyticum, Mycoplasma hominis, Treponema pallidum, Hepatitis B, Herpes simplex virus, Human Immunodeficiency Virus, Human papillomavirus, Molluscum, Phthirius pubis, Sarcoptes scabiei and Trichomonas vaginalis.

In yet an embodiment of the invention the infectious diseases are selected from the group consisting treatable gastro-intestinal infectious agents e.g. Shigella, E. Coli, Campylobactor, Vibrio cholerae bacteria, Cryptosporidium parvum, Salmonella bacteri and Salmonella typhi bacteria.

In yet an embodiment of the invention the infectious diseases are selected from the group consisting gastro-intestinal infectious agents not treatable with antibiotics e.g. rotaviruses, noroviruses, adenoviruses, sapoviruses, and astroviruses.

In a further embodiment of the invention the infectious diseases are selected from the group consisting of blood related diseases that are subject to screening e.g. in blood banks: Hepatitis A, Hepatitis E, Malaria, Chagas Disease, Babesiosis, Leishmaniasis, Simian foamy virus, Creutzfelt-Jacob Disease (vCJD), Creutzfeldt-Jakob Disease (CJD), Cytomegalovirus (CMV) and Epstein-Barr Virus.

In one embodiment of the invention the infectious diseases are selected from the group consisting of bacterial able to cause bacterial meningitis, Neisseria meningitides, Streptococcus pneumoniae, Listeria monocytogenes, Pseudomonas aeruginosa, Staphylococcus aureus, Streptococcus agalactiae and Haemophilus influenza.

Accordingly it is object of the present invention to make a species specific diagnosis, because selection of a specific antigen enables the skilled addressee to differentiate between various species of e.g. Mycobacterium.

Prognosis

In one embodiment, the method may be used for predicting the prognosis of individuals diagnosed with various immunological conditions, such as infections. When used in patient prognosis the method according to the present invention may help to predict the course and probable outcome of the immunological condition, such as infections, thus assisting the skilled artisan in selecting the appropriate treatment method and assess the probable effect of a certain treatment for the condition.

Monitoring

In one embodiment, the method may be used for monitoring individuals diagnosed with infections. When used in patient monitoring the method according to the present invention may help to assess efficacy of treatment during and after termination of treatment e.g. monitoring and predicting possible recurrence of the infection.

The possibility to monitor therapy efficacy by the present invention is particularly relevant because (using infection with M. tuberculosis as an example)

a) it is easy to perform by a simple blood draw instead of currently available methods like sputum microscopy, mycobacterium culture, X-ray or other methods

b) it is more reproducible compared to sputum microscopy, mycobacterium culture, X-ray or other methods

c) it is in-expensive, compared sputum microscopy, mycobacterium culture, X-ray or other methods, invasive surgery procedures involved in a biopsy, e.g. if an extra-pulmonary tuberculosis is suspected, or a broncoscopy, if the patient is sputum negative

d) it reduces the number of false negative test results and indeterminate test results and is more sensitive compared to the classical 37° C. assays for tuberculosis based on RD1 overlapping peptides;

e) it may distinguish between active and latent tuberculosis while the other immune assays only distinguish infection or no infection.

Screening

In one embodiment, the method according to the present invention is used for screening purposes. I.e. it is used to assess subjects without a prior diagnosis of the relevant infection(s) by measuring the level of IP-10 according to the invention and correlating the level measured to a pre-specified level, indicating the presence or absence of various infections (e.g. infection with M. tuberculosis). In another embodiment, the method according to the present invention is used for screening purposes. I.e., it is used to assess subjects without a prior diagnosis of the relevant infection(s) but at risk of reactivation of latent disease by measuring the level of IP-10 according to a pre-specified level indicating the invention and correlating the level measured to the presence or absence of various infections (e.g. infection with M. tuberculosis).

As stated previously the present invention discloses a method for simultaneous screening for at least two infectious diseases.

In another embodiment of the present invention, the method can be used to screen blood from blood-donors for various diseases such as but not limited to infection(s) caused by e.g. parasites or vira.

Contact Tracing

In preferred embodiments, the method according to the present invention may be used for diagnosis of subjects exposed to various infections, such as M. Tuberculosis. When used in contact tracing the method according to the present invention may help to determine the presence of infections, such as infection with M. tuberculosis.

In other embodiment, the method according to the present invention may be used for diagnosis of subjects exposed to contagious cases in outbreaks of highly contagious infections, such as, but not limited to Tuberculosis, Corona viruses (e.g. Severe Acquired Respiratory Syndrome), Influenza, Ebola or Marburg virus. In the case of tuberculosis: When used in contact tracing the method according to the present invention may help to determine the presence of infection, usually accomplished by evaluating the TST or the currently available IFN-γ release assay.

Enhanced Case Finding

In preferred embodiments, the method according to the present invention may be used for diagnosis of various diseases, such as infections. When used in enhanced case finding the method according to the present invention may help to determine the presence of infections, such as but not limited to microscopy negative TB which is otherwise difficult to diagnose due to the lack of microbiological evidence of infection but which is usually accomplished by evaluating clinical symptoms, response to treatment, and lack of alternative diagnoses or by time-consuming assays (weeks) such as sputum culture.

Prevalence Studies

In preferred embodiments, the method according to the present invention may be used for studying the prevalence of various immunological states, such as but not limited to infections in populations of interest such as children, HIV positive immigrants, refugees, health care workers, school children, prisoners, laboratory technicians. When used in prevalence studies, the method according to the present invention may help to determine the presence of an infection, such as latent and active TB in a population, usually accomplished by the TST.

Research Purposes

In one embodiment, the method according to the present invention may be used by research institutions when screening for potential new antigens derived from a micro organism selected from the group consisting of Mycobacteria, gram positive bacteria, gram negative bacteria, Listeria, enterococci, Neisseria, vibrio, treponema (Syphilis), Borrelia, leptospires, Clamydia, retroviruses (SIV, HIV-1 and HIV-2), corona viruses such as Severe Acute Respiratory Syndrome (SARS) and NL-63, Cytomegalovirus , rotaviruses, metapneumovirus, respiratory syncytium virus (RSV), poxviruses, Ebstein barr virus, enterovirus, morbillivirus, rhabdoviruses (rabies). Rubivirus (rubella), flaviviruses (dengue, yellow fever), herpes viruses, varicellea-zoster virus, Hepatitis C and B, Leishmania, Toxoplasma gondii, trypanosoma, Plasmodium (falciparum, vivax, ovale, malaria), pneumocystis cariini (PCP) and various nematodes, trematodes, these antigens can be e.g. lipids, polysaccharide molecules, proteins and peptides. When used for laboratory research purposes the method according to the present invention may help to determine immune reactivity to the examined antigen, protein or peptide applicable in development of vaccines and diagnostic tests.

Several antigen molecules like for instance peptides are identified as species specific or disease-specific, but their ability to induce T cell reactivity in vivo is difficult to determine due to a lack of sensitive markers. The level of immune signalling molecule(s) determined after stimulation at hyperthermic temperature with such candidate antigens can be used to screen for and identify potentially interesting new antigens or molecules. More specifically in the case of antigens derived from M. tuberculosis, C. trachomatis, HIV-1 or HCV; the method may be used by research institutions when testing the immunogenicity of these antigens i.e. as a measure of T cell reactivity for the development of e.g. vaccines.

It should be understood that any feature and/or aspect discussed herein in connection with the determination according to the invention apply by analogy to the “diagnosis”, “prognosis”, “monitoring”, “screening”, “research purposes”, “contact tracing”, “enhanced case finding” and “prevalence studies” according to the invention and visa versa.

The present invention provides a methods with improves testing and diagnosing. As seen in the Example the increased incubation temperature results in an increased responsiveness in the lowest of the responders. These surprising findings show that the hyperthermic incubation method can boost the response from otherwise “false negative” low responders leading to fewer false negative test results and thus increase the sensitivity compared to the traditional 37° Celsius method. Furthermore, with 39° C+IL7 and anti-IL-10 very few donors show low responsiveness to the mitogen. This is an important finding as it indicates that we are able to make cells from patients with immuno-suppression (HIV, cancer, various types of medical treatment) responsive, which decreases the amount of indeterminate test results and thus increases cost-effectiveness.

Vaccination

One aspect of the present invention relates to a method, wherein the test-antigen dependent immune signalling molecule response above the reference-level indicate that the mammal has previously encountered the antigen or previously encountered other antigens generating cross reactivity to the antigen because of a vaccination against any micro-organism mentioned herein.

Response to a vaccine based on non-viable material may result in low levels of antigen-specific immune signalling molecules and because the present method lead to an improved immune response (such as higher release of the immune signalling molecule or lower background levels) it may be used to detect vaccine responses in preclinical, clinical trials, and subsequently in a routine setting.

In another preferred embodiment the present invention the method is useful for monitoring the effect of a vaccine. By monitoring the IP-10 response in according with the teachings of this invention using antigens comprised in the vaccine, it is possible to determine the effect of the vaccination. Such a measure is important when evaluating the need for revaccination or likelihood of potential benefit of the vaccine.

Thus the present invention also relates to a method wherein said test-antigen specific cell-mediated immune response is used to detect a vaccination response.

Microorganism

According to the present invention the infections may be caused by a micro organism, such as but not limited to bacteria, parasites, fungi, viruses, prions, and/or viroids.

In a presently preferred embodiment the micro organism is selected from the group consisting of Mycobactiera, gram positive bacteria, gram negative bacteria, Listeria, enterococci, Neisseria, vibrio, treponema (Syphilis), Borrelia, leptospir, Chlamydia, retroviruses (SIV, HIV-1, HIV-2), Cytomegalovirus , poxviruses, Ebstein barr virus, enterovirus, morbillivirus, rhabdoviruses (rabies). Rubivirus (rubella), flaviviruses (dengue, yellow fever), herpes viruses, varicella-zoster virus, Hepatitis C and B, Leishmania, Toxoplasma gondii, trypanosoma, Plasmodium (falciparum, vivax, ovale, malaria), pneumocystis cariini (PCP), Coronavirus (e.g. Severe Acquired Respiratory Syndrome (SARS)), Ebola or Marburg and various nematodes, trematodes.

In an even more preferred embodiment the microorganism is selected from the group consisting of Mycobacteria, Leishmania, Chlamydia and Cytomegalovirus

In the case wherein the infection is or were caused by Mycobacteria, said Mycobacteria belongs to the M. tuberculosis complex organisms (M. tuberculosis, M. bovis and M. africanum), and Mycobacteria where the region of difference (RD1) has not been deleted (M. kansasii, M. szulgai, M. marinum, M. flavescens, M gastrii) or Mycobacteria pathogenic to humans (M. avium, M. lepra or other non-tuberculous mycobacteria)

Thus in one presently preferred embodiment the Mycobacteria is M. tuberculosis.

Tuberculosis

Tuberculosis (commonly abbreviated as TB) is an infectious disease caused by the bacterium Mycobacterium tuberculosis (M. tuberculosis), which most commonly affects the lungs (pulmonary TB) but can also affect all other organs in the body e.g. the central nervous system (meningitis), lymphatic system, circulatory system (miliary tuberculosis), genitourinary system, bones and joints. Infection with M. tuberculosis can also remain asymptomatic which is commonly known as latent, dormant or sub-clinical TB infection. From this stage the infection can progress to active disease which is often due to immunodeficiency, caused by e.g. HIV co-infection or immunosuppressive treatment.

In a presently preferred embodiment, the present invention relates to a method of diagnosing and monitoring various e.g. distinct presentations of tuberculosis: active tuberculosis disease, active microscopy positive or microscopy negative TB infection, latent tuberculosis infection, and recent tuberculosis infection.

The method is based on the evaluation of the production of immune signalling molecules such as IP-10 by antigen-specific T lymphocyte in interaction with antigen presenting cells (e.g. monocytes/macrophages) responding to selected peptide sequences of secretory proteins of M. tuberculosis. These peptide sequences have been selected for their immunogenicity and their specificity, and potentially other peptides can be used similarly.

The method can be used for diagnosing active tuberculosis disease, for diagnosing a recent infection in healthy contacts of a patient with a sputum-positive pulmonary tuberculosis, for diagnosing healthy with latent infection, for monitoring the response to treatment in the case of pulmonary and extra-pulmonary tuberculosis and to discriminate between latent infection and active tuberculosis disease state

Mammal

Reference to a “mammal” or a “subject” includes a human or non-human species including primates, livestock animals such as but not limited to sheep, cows, pigs, horses, donkey, goats, laboratory test animals and companion animals. The present invention has applicability, therefore, in human medicine as well as having livestock and veterinary and wild life applications.

### EXAMPLES

In the following examples the use of hyperthermic conditions and/or presence of IL7 and anti-IL-10 for augmenting a test-antigen specific cell-mediated immune response are demonstrated using stimulation with antigens from Mycobacterium tuberculosis as an example.

General Methods

Donors

For preliminary study (example 1), blood samples were collected from healthy donors employed at the study site and from patients suspected of or starting treatment for TB attending the outpatient clinic at the Department of Infectious Diseases, Hvidovre Hospital.

For the BCG vaccine study (examples 2-5 and 7), blood samples were collected from 35 healthy donors employed at the study site, the outpatient clinic at the Department of Infectious Diseases or in the Clinical Research Centre at Hvidovre Hospital, Denmark.The 35 donors donors were grouped into BCG-vaccinated or non-BCG vaccinated and unless the donors knew their BCG-vaccination status, grouping was done by birth year as BCG vaccination of Danish children was discontinued in 1975.

For the TB diagnosis study (example 6-7) blood was collected from patients suspected of or starting treatment for TB attending the outpatient clinic at the Department of Infectious Diseases, Hvidovre Hospital, Denmark.

Reagents and Equipment

For the BCG vaccine study TB10.4 peptides were used as antigens (JPT Peptide Technologies GmbH, Berlin, Germany) and lectin from Phaseolus vulgaris as mitogen (PHA; Sigma-Aldrich Corp, Missouri, USA). As additional stimulants (immune modulators), we used recombinant human IL-7 (R&D Systems Inc., Minneapolis, USA) and monoclonal antibody towards human Interleukin 10 (anti-IL-10; MBL Intl., Massachusetts, USA). For temperature incubation we used incubators, water baths and heating blocks.

For the TB diagnosis study, QuantiFERON TB Gold In tube blood collection tubes were used. These consist of an antigen tube containing TB specific antigens (ESAT-6, CFP-10, TB7.7), a mitogen tube containing PHA and a nil tube. For the BCG vaccine study and the TB diagnosis study, an incubator was used for incubation at 37° C. while a water bath placed within an incubator was used for incubation at 39° C. Temperatures were checked at least 4 times during each incubation round with at least 2 hours interval and did not vary with more than 0.2° C.

Blood Collection and Incubation for Biomarker Measurements

Blood was collected in heparinized tubes (BD Vacutainer Systems, Plymouth, UK) or in QuantiFERON blood collection tubes for the TB diagnosis study.

For the preliminary studies 1 ml of blood was transferred to a nunc cryotubes (Thermo Fisher Scientific, Roskilde, Denmark), whereupon we followed a series of steps for incubation optimization. Step 1: Titration of PHA and TB10.4 from 0.16 to 20 μg/ml and 0.04 to 20 μg/ml respectively. Step 2: Incubation of samples at different temperatures in 3 consecutive trials: i) 20, 30. 37, 39, 41 and 43° C., ii) 37, 38, 39, 40, 41 and 42° C. and iii) 37.0, 37.3, 37.9, 38.5, 39.0, 39.5 and 39.9° C. for 18 hours of incubation each. Step 3: Incubation of samples for 0, 6, 9, 12, 15, 18, 24 and 48 hours at 37 and 39° C. respectively. Step 4: Incubation of samples with IL-7 (2.0 ng/ml), anti-IL-10 (1.0 μg/ml) and both IL-7 and anti-IL-10 for 18 hours at 37 and 39° C. respectively and with an addition of 2 mg/ml dextrose to each tube. Concentrations of IL-7 and anti-IL-10 were adopted from the literature [Feske Clin Vacc Immunol 2008, Denis Clin Vacc Immunol 2007].

For the BCG vaccine study 1 ml of blood was transferred to a nunc cryotubes (Thermo Fisher Scientific, Roskilde, Denmark) and the relevant stimulant was added. For each trial, three samples were incubated with different stimulant:

antigen tubes with TB10.4 peptide suspension, mitogen tubes with PHA suspension and nil tubes with PBS for background measurements with either no additional cytokine, with IL-7 (2.0 ng/ml), with anti-IL-10 (1.0 μg/ml) or with both IL-7 and anti-IL-10 for 18 hours at 37 and 39° C. respectively giving a total of 24 tubes per donor. Dextrose was added to all tubes to a concentration of 2 mg/ml

For the QuantiFERON study, 1 ml of blood was transferred to each blood collection tube (nil, antigen and mitogen). These where then incubated with no additional stimulant or with IL-7 (2.0 ng/ml) and anti-IL-10 (1.0 μg/ml) at 37 or 39° Celsius for 18 hours.

Measurements

For preliminary and BCG vaccine study, IP-10 measurements were done directly following plasma harvesting whereupon samples were frozen at −80° Celsius. After 6 months, BCG vaccine study samples were thawed and IFN-γ measurements were done. For the TB diagnosis study plasma was harvested directly following incubation and frozen at −80° Celsius.IP-10 and IFN-γ measurements where done simultaneously after 6 to 8 months. For IP-10 measurements, samples were diluted 1:9 in assay diluents and run in duplicates using a sandwich ELISA with a standard curve with linearity from 2000 pg/ml down to 31.8 pg/ml. In brief NUNC MaxiSorb plates were coated over night with murine monoclonal mAbs specific for human IP-10. Plates were washed and blocked using buffer with 10% Bovine serum albumin. Plasma samples were then added in duplicates and incubated for 1 hour at 37° Celsius. Then plates were washed and murine detection mAb coupled with horse radish peroxidise enzyme was added, and allowed to incubate at 37° Celsius for 45 minutes. Plates were then washed ×7 and TMB substrate was added. The plate developed for 15 minutes, whereafter the reaction was stopped by the addition of 1M H2SO4. IFN-γ levels were measured using the commercial QuantiFERON-TB Gold (QFT-IT) ELISA. In order to better quantify the levels of IFN-γ, the standard curve for the QFT-IT ELISA was extended, giving linearity between 800 and 12.5 pg/ml. In all other aspects, manufacturer's instructions were followed. One IU equals 50 pg of IFN-γ.

In the data presented, background levels of biomarkers (nil) are subtracted unless otherwise indicated (raw values). Antigen dependent levels of biomarker are designated “a” (i.e. aIFN-γ and aIP-10) while mitogen induced biomarker levels are designated “rn” (i.e. mIFN-γ and mIP-10).

Leukocyte levels and differential counts were measured at the Clinical Laboratory at Hvidovre Hospital.

Statistical Analysis.

Data were analyzed using SAS 9.2 (SAS Institute, Cary, N.C., USA). Since no parameters were normally distributed all tests were done using non-parametric tests (Wilcoxon signed-rank test and Spearman's test for correlations). A synergicstic effect was defined as a greater effect of two stimuli (anti-IL-10, IL-7 and temperature) together than the added value of each of the stimuli alone. All tests were done two-sided and results with a p-value 0.05 were considered significant.

Ethical Considerations.

Permission to conduct the study was obtained from the Ethical Committee of the Municipality of Copenhagen. All study participants gave written informed consent to participate and were free to withdraw from the study at any time.

### Example 1

Hyperthermic temperatures augment the IP-10 and IFN-γ responses.

We investigated whether we were able to augment the IP-10 and IFN-γ responses by increasing the incubation temperature.

Results

FIG. 1 is a spaghetti diagram showing two representative individuals from the preliminary study, and we were able to reproduce the findings of these experiments for 11 additional individuals. We found that the signal was consistently and significantly increased at temperatures up to 39.5° Celsius, while at higher temperatures we observed a steep decline in the response.

In conclusion, incubation at hyperthermic conditions results in increased IP-10 and IFN-γ responses when whole blood from BCG vaccinated individuals is stimulated with BCG specific antigens when compared to incubation at 37° Celsius.

Incubation at high temperature has great potential as an easy method to reduce the number of false negative and indeterminate test results and thus improve the sensitivity and cost-effectiveness of tests relying on determination of the presence/level of biomarkers such as IP-10 and/or IFN-γ induced after antigen-specific stimulation.

### Example 2

(BCG Vaccine Study)

Division of donors into non-responders and responders by measuring the levels of plasma IP-10 induced by stimulation of whole blood with M. tuberculosis specific antigens (TB10.4).

A total of 35 donors were tested: 16 BCG-vaccinated donors who were thus likely to respond to TB10.4 antigens and 19 un-vaccinated donors who were less likely to respond to TB10.4 antigens. Whole blood was drawn and incubated for 18 h at 37° Celsius and 39° Celsius respectively with a cocktail of peptides from the antigen TB10.4.

10 Based on the preliminary adjustments we chose to compare standard 37° Celsius incubation with the new 39° Celsius incubation. We chose the apparently inferior 39° Celsius to 39.5° Celsius as we wanted proof of concept data on a stable system.

Results

We collected whole blood from 34 healthy donors (one BCG-vaccinated donor was excluded due to failed venopuncture). Levels of IP-10 in the plasma of whole blood samples incubated with TB10.4 were measured by an in-house ELISA (as described above).

After measuring IP-10 we divided the participants into two groups: responders and non-responders defined by who could and who could not generate an in-vitro immune response (arbitrarily defined with an IP-10 production >500 pg/ml towards peptides from the protein TB10.4 when incubated at standard incubation temperature 37° C.) (see FIG. 2 and table 1)

TABLE 1

| | Non-responders | Responders | |
|---|---|---|---|
| | (n = 11) | (n = 23) | P |
| Age, median (range) | 30 (26-48) | 39 (26-54) | 0.09 |
| WBC, median (range) | | | |
| Total | 6.83 (3.94-10.94) | 6.06 (4.03-8.63) | 0.24 |
| Neutrophils | 3.56 (1.38-5.95) | 3.13 (1.71-6.11) | 0.39 |
| Lymphocytes | 2.07 (1.84-3.91) | 1.92 (1.37-3.30) | 0.05 |
| Monocytes | 0.44 (0.30-0.85) | 0.47 (0.23-0.82) | 0.81 |
| Eosinophiles | 0.13 (0.07-0.44) | 0.09 (0.03-0.49) | 0.17 |
| Basophiles | 0.02 (0.01-0.06) | 0.03 (0.01-0.06) | 0.57 |

There was no significant difference in either age or white blood cell counts between non-responders and responders.

### Example 3

Hyperthermic incubation with or without IL-7 and/or anti-IL-10 improve the IP-10 response.

We validated the effect of hyperthermic incubation on the IP-10 response by testing the samples from above mentioned 34 responders and non-responders.

Next we tested if the presence of the cytokine IL-7 during incubation would improve the response. We also tested whether blockade of the inhibitory cytokine IL-10 would influence the IP-10 response by inhibiting a known cardinal inhibitor in the system (i.e. inhibiting an inhibitor).

Results

FIG. 3A-D shows the IP-10 responses in 8 columns. The initial 4 columns represent incubation at 37° Celsius, the latter 4 incubation at 39° Celsius. Blood from the study participants was divided into aliquots and subjected to all the various culture conditions, i.e. columns can be directly compared. Background levels (nil) are subtracted from the antigen and mitogen responses.

FIG. 3A shows the influence on biomarker levels of different incubation conditions in non-responders (non-resp.). We see that there are few significant differences in responsiveness between the different culture conditions, but interestingly two of the non-responders were brought to respond at 39° Celsius. These two, who would be non-responders in currently used methods, were the only BCG-vaccinated persons in the non-responder group and they were also the two with the “highest” IP-10 signals of the non-responders in FIG. 2. These surprising findings show that the hyperthermic incubation method can boost the response from otherwise “false negative” low responders and thus increase the sensitivity compared to the traditional 37° Celsius method.

FIG. 3B show the results for the responders. We see a striking improvement in IP-10 responsiveness at 39° Celsius compared to 37° Celsius. Adding IL-7, blocking IL-10, and especially the combination of both further improves the IP-10 signal. Very few thus remain “low responders” at 39° Celsius.

5 In FIG. 3C we examine the potential problems with increased background IP-10 levels with the various modifications of the incubation. It is evident that especially the high temperature does lead to a larger degree of noise in the system, but the levels are negligible compare to the striking improvements seen in responses from responders (note the differences in axis scale).

In FIG. 3D we compare IP-10 responses to the unspecific PHA mitogen stimulation. We reproduce the findings from 3B, thus demonstrating that the findings are not only present when stimulating with specific peptides, but also with unspecific stimulation.

Thus, when testing the 34 responders and non-responders we found that hyperthermic incubation at 39 Celsius improves the IP-10 response as also observed in Example 1.

From the graphs it seems that the increase in IP-10 responsiveness to mitogen was lower compared to responsiveness to antigen stimulation, however this is an artefact as the upper range of the assays are 20000 pg/ml, and 800 pg/ml for IP-10 and IFN-γ respectively; concentrations above these limits are thus inaccurate.

In conclusion, the findings show that the hyperthermic incubation method can increase the magnitude of IP-10 released and reduce the number of false negative and indeterminate test results and thereby increase the sensitivity compared to the traditional 37° Celsius method. Furthermore, adding IL-7, blocking IL-10, and especially the combination of both increases the IP-10 response when whole blood from BCG vaccinated individuals is stimulated with BCG specific antigens.

### Example 4

Hyperthermic incubation and the presence of IL-7 and/or anti-IL-10 improve the IFN-γ response.

We validated the effect of hyperthermic incubation on the IFN-γ response by testing the samples from 34 responders and non-responders and analysed if adding IL-7 or blocking IL-10 would improve the IFN-γ response.

Results

In FIG. 4A we see that the IP-10 non-responders were also IFN-γ non-responders. We were pleased to be able to reproduce the findings from FIG. 3A with the same two BCG-vaccinated non-responders suddenly responding to the TB-10.4 antigens by 39° C. incubation.

FIG. 4B shows the effect of the incubation conditions on IFN-γ levels of TB10.4 responders and we were surprised to find that the striking improvements seen for IP-10 were not as pronounced for IFN-γ in this vaccine recall test system. There were no significant improvement with increased temperature alone when analyzed by non-parametric analysis, but what we did find was that increased incubation temperature resulted in an increased responsiveness in the responders with the lowest response.

FIG. 4C illustrates that 39° Celsius incubation temperature leads to significantly lower background levels of IFN-γ. Interestingly this is the opposite effect seen for IP-10 showing that although the improvements in antigen-specific IFN-γ responsiveness are not as pronounced as for IP-10, the lower background levels can lead to a better signal to noise ratio in the system (elaborated in FIG. 5).

In FIG. 4D we compare INF-γ responses to the unspecific PHA mitogen stimulation and the findings are similar to the findings in FIG. 3D. It is, however, noteworthy that incubation at 39° Celsius alone increases the response significantly. Furthermore, with 39° Celsius+IL7 and anti-IL-10 we see very few donors with low responsiveness to the mitogen.

Thus, when testing the 34 responders and non-responders we found a similar beneficial effect on the INF-γ response of incubation at 39° Celsius.

From the graphs it seems that the increase in INF-γ responsiveness to mitogen was lower compared to responsiveness to antigen stimulation, however this is an artefact as the upper range of the assays are 20000 pg/ml, and 800 pg/ml for IP-10 and IFN-γ respectively; concentrations above these limits are thus inaccurate.

In conclusion, incubation at hyperthermic conditions results in increased responsiveness in the lowest responders and decreased backgrounds levels when whole blood is stimulated with BCG specific antigens. Furthermore adding IL-7 and blocking IL-10 augmented the INF-γ signal. Very few thus remain “low responders” at 39° Celsius.

Thus one embodiment of the present invention relates to a method that makes cells from patients with immuno-suppression (HIV, cancer, various types of medical treatment) responsive by incubation at 39° Celsius. This would lead to better performance of immunodiagnostic tests (i.e. fewer indeterminate test results) in these challenging patients groups.

### Example 5

Synergistic effects of hyperthermic incubation and the presence of IL-7 and/or anti-IL-10 on the production of IP-10 and INF-γ.

Combining all the stimuli gave superior levels of both IP-10 and IFN-γ compared to all other combinations after both antigen and mitogen stimulation. Except for the combination of hyperthermic incubation (temperature) and addition of anti-IL-10, we found a synergistic effect on IP-10 production of using hyperthermic incubation together with addition of IL-7 and/or anti-IL-10 after both antigen and mitogen stimulation (p<0.05 for all, table 2). The synergistic effect observed on background IP-10 levels was due to increased background IP-10 levels when applying stimuli. For IFN-γ, a synergistic effect was observed when using hyperthermic incubation together with IL-7 and anti-IL-10 after antigen stimulation. Also, adding IL-7 and using hyperthermic incubation with or without addition of anti-IL-10 synergistically lowered background IFN-γ levels

We found no correlation between monocyte or lymphocyte count with levels of antigen dependent or mitogen induced production of neither IP-10 nor IFN-γ (data not shown).

TABLE 2
Synergy between hyperthermic incubation (temperature) and
anti-IL-10, IL-7 and both anti-IL-10 and IL-7

| | IP-10 | | | IFN-γ | | |
|---|---|---|---|---|---|---|
| | Antigen | Mitogen | Nil | Antigen | Mitogen | Nil |
| | (responders) | (all) | (all) | (responders) | (all) | (all) |
| Temperature + | YES | NO | YES | NO | NO | NO |
| anti-IL-10 | (p = 0.01) | (p = 0.07) | (p = 0.001) | (p = 0.16) | (p = 0.20) | (p = 0.07) |
| Temperature + | YES | YES | YES | NO | NO | YES |
| IL-7 | (p = 0.0016) | (p = 0.0001) | (p = 0.001) | (p = 0.48) | (p = 0.61) | (p < 0.0001) |
| Temperature + | YES | YES | YES | YES | NO | YES |
| anti-IL-10 + | (p = 0.0001) | (p = 0.0001) | (p = 0.001) | (p = 0.0082) | (p = 0.48) | (p < 0.0001) |
| IL-7 | | | | | | |

### Example 6

Hyperthermic incubation increases responsiveness of both IFN-γ and IP-10 to TB specific test-antigens for diagnosis of TB.

We then compared the IFN-γ and IP-10 levels respectively after incubation of the QuantiFERON TB Gold In tube test tubes at 39° Celsius with and without adding IL-7 and blocking IL-10 to the normal incubation at 37° Celsius. Blood was collected from 9 patients suspected of tuberculosis suspected of or starting treatment for TB. Levels of IP-10 in the plasma were measured by an in-house ELISA (as described above) and levels of IFN-γ were measured using the QuantiFERON TB Gold ELISA (as described above).

Results

FIG. 5 A-C and 6A-C show the IP-10 responses in 4 columns for IP-10 and IFN-γ respectively. The initial 2 columns represent incubation at 37° Celsius, the latter at 39° Celsius with and without IL-7 and anti-IL-10 respectively. Background levels (nil) are subtracted from the antigen and mitogen responses.

In FIG. 5A and 6A we examine the potential problems with increased background levels with the various modifications of the incubation. We found no significant increases in background IP-10 levels between the different incubation conditions. Interestingly, but similar to the findings for vaccine specific responses, background levels of IFN-γ were lower when incubating at 39° Celsius.

FIG. 5B and 6B show the IP-10 and IFN-γ levels respectively in the antigen tubes. We see a striking improvement in TB specific antigen responsiveness for both IP-10 and IFN-γ at 39° Celsius compared to 37° Celsius especially when adding IL-7 and blocking IL-10. The effect is even more pronounced that the effect seen for vaccine specific responses (in FIGS. 3 & 4). Very few thus remain low or non-responders at 39° Celsius.

In FIG. 5C and 6C we compare IP-10 and IFN-γ responses respectively in the

PHA mitogen tubes. We reproduce the findings from 5B and 6B, thus demonstrating that the findings are not only present when stimulating with specific peptides, but also with unspecific stimulation.

Thus, when testing suspected TB patients in the QuantiFERON TB Gold In tube test system, we found that hyperthermic incubation at 39° Celsius improves both the IP-10 and the IFN-γ responses as also observed in the vaccine recall examples above.

When interpreted into a test result, 1 patient had a negative (antigen response below 17.5 pg/ml and mitogen response above 25 pg/ml) and 2 had an indeterminate QuantiFERON result (antigen response below 17.5 pg/ml and mitogen response below 25 pg/ml) when incubating at 37° Celsius, even when adding IL-7 and anti-IL-10. The patient with a quantiferon negative result remained negative regardless of incubation condition. One patient with an initial Quantiferon indeterminate result converted to positive with very high levels of both IFN-γ when incubating at 39° Celsius. The other patient with an indeterminate quantiferon result was revealed as quantiferon negative after incubation at 39° Celsius with consistently low IFN-γ responses after antigenic stimulation, but responding strongly to mitogen stimulation only after incubation at 39° Celsius. The same pattern was found for IP-10 for these patients.

In conclusion, incubation at hyperthermic conditions results in increased IP-10 and IFN-γ responsiveness to M. Tuberculosis specific antigens, also in the lowest responders. Furthermore, it decreased backgrounds IFN-γ levels, also when whole blood is stimulated with antigens specific for M. tuberculosis. Adding IL-7 and blocking IL-10 augmented both IP-10 and IFN-γ levels. Very few thus remain “low responders” at 39° Celsius.

These findings show, that the hyperthermic incubation method can reduce the number of false negative and indeterminate test results and thereby increase the sensitivity compared to the traditional 37° Celsius method. The method has been exemplified for vaccine response and diagnostic tests based on antigen specific stimulation and can be extended to other indications such as but not limited to cancer diagnostics and cancer monitoring by changing the specificity of antigen

### Example 7

The method improves the separation between nil and antigen and thereby the signal-to-noise ratio for antigens specific responses.

To better illustrate why the present method improves the diagnostic information of IFN-γ, the raw values of antigen and mitogen have been plotted compared to nil for all the different culture conditions, after vaccine specific antigen stimulation (FIG. 7). Better separation between nil and antigen means a better signal to noise. It is evident that the 39° Celsius+IL-7 +anti-IL-10 cocktail leads to higher antigen response and lower nil response and thus a perfect separation between nil and antigen.

To fully appreciate the improved separation we have calculated the response ratio (i.e. the stimulated level divided with the nil level) for both stimulation with mitogen and the M. Tuberculosis-specific (ESAT6,CFP10,TB7.7) antigens. In FIG. 8A IFN-γ nil values are presented, and the response ratios with antigen and mitogen is presented in 8B and 8C respectively. In FIG. 9A IP-10 nil values are presented, and the response ratios with antigen and mitogen is presented in 9B and 9C respectively. Again it is evident that the 39° Celsius (optionally with IL-7 and anti-IL-10 cocktail) leads to higher antigen response and lower nil response and thus a perfect separation between nil and antigen.

### FIGURE LEGENDS

FIG. 1

The impact of incubation temperature on IP-10 and IFN-γ responsiveness. Whole blood was drawn and incubated 18 h at 25, 35, 37, 38, 38.5, 39, 39.5, 40, 41 and 43° Celsius with a cocktail of peptides from the antigen TB10.4 or PHA. In some experiments, fewer temperatures were tested. Data shown are from one experiment with two representative donors. Concentrations are in pg/ml.

FIG. 2

IP-10 responses in plasma from in 34 healthy donors ordered from lowest to highest responder. Whole blood was drawn and incubated for 18 h at 37° Celsius with a cocktail of peptides from the antigen TB10.4. Donors who responded with >500 pg/ml IP-10 were considered responders

FIG. 3

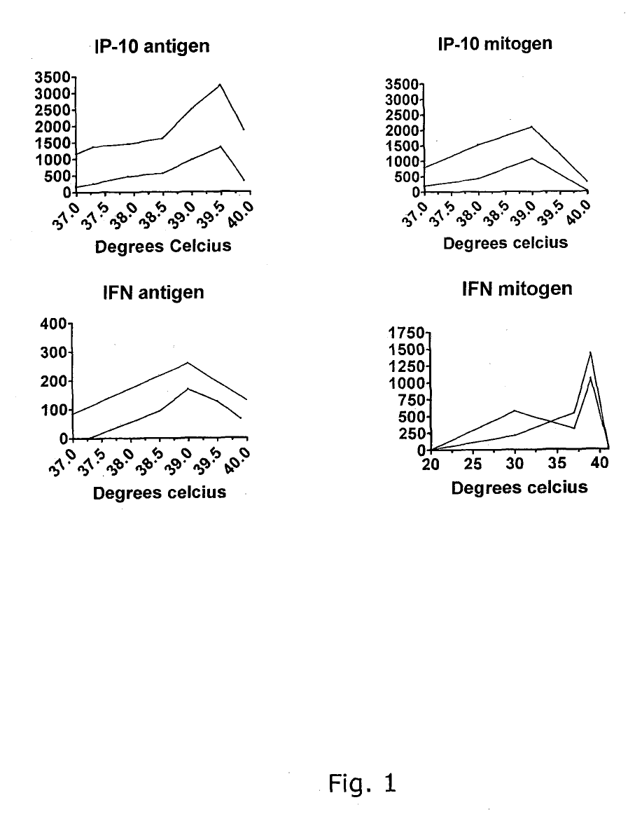

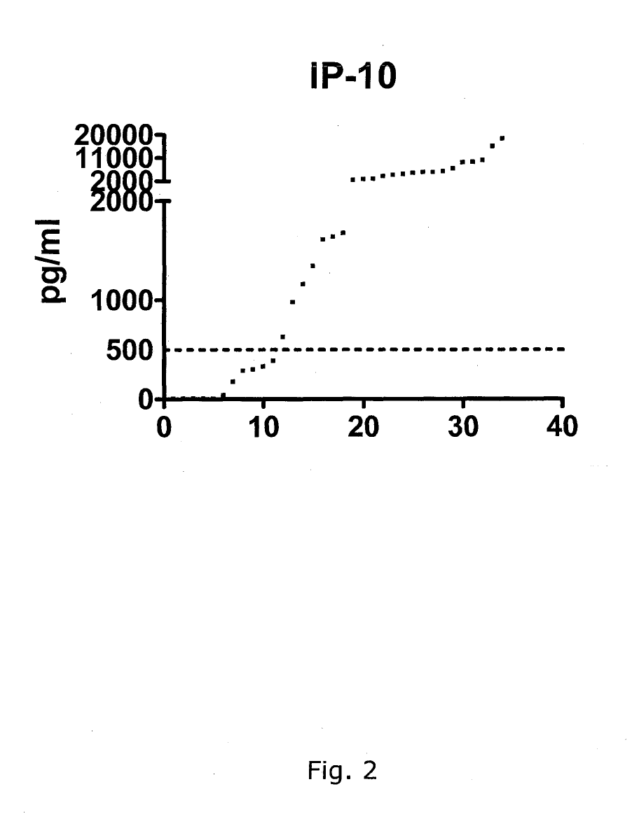

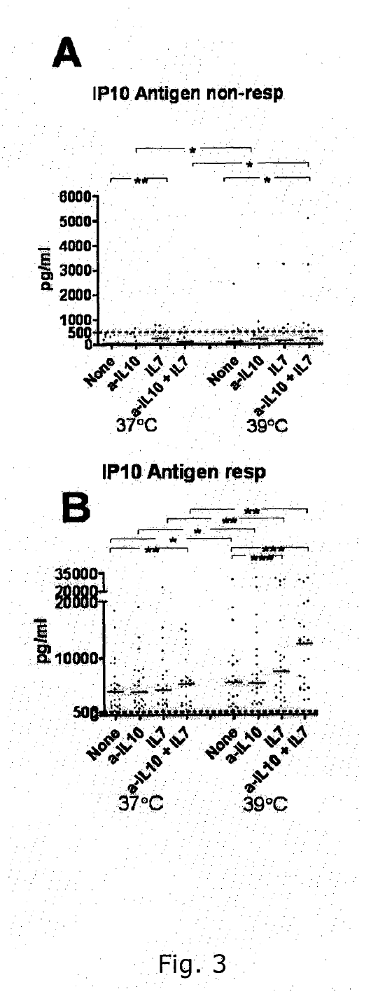

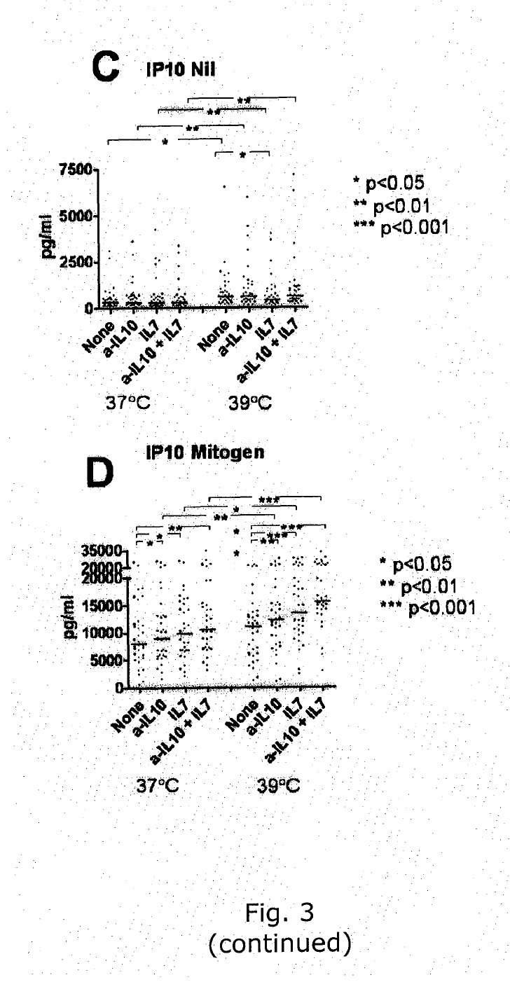

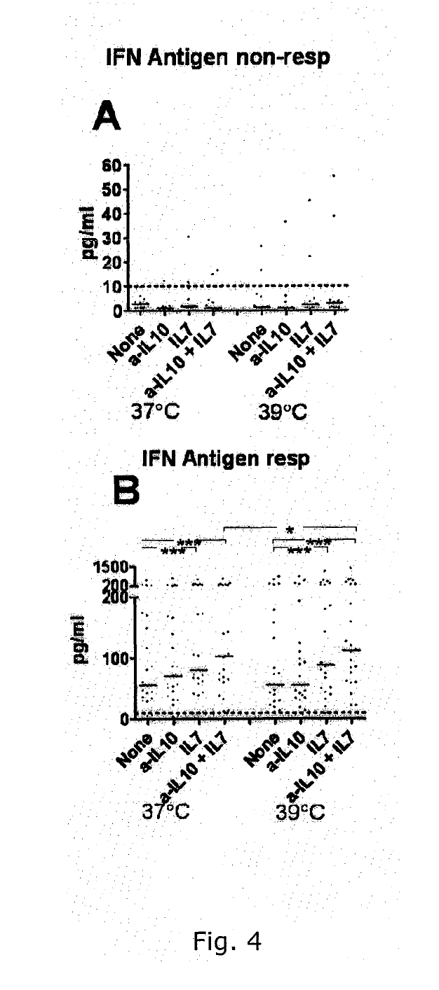

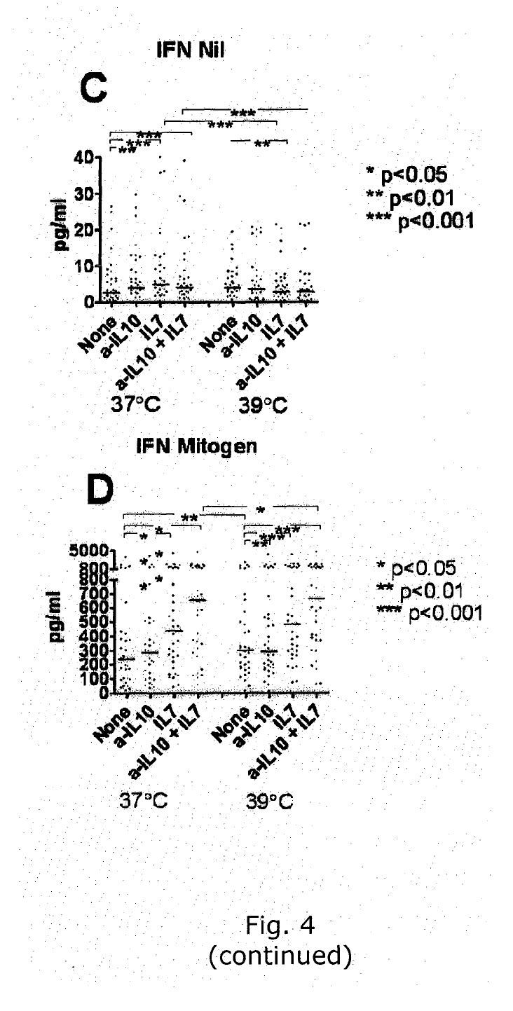

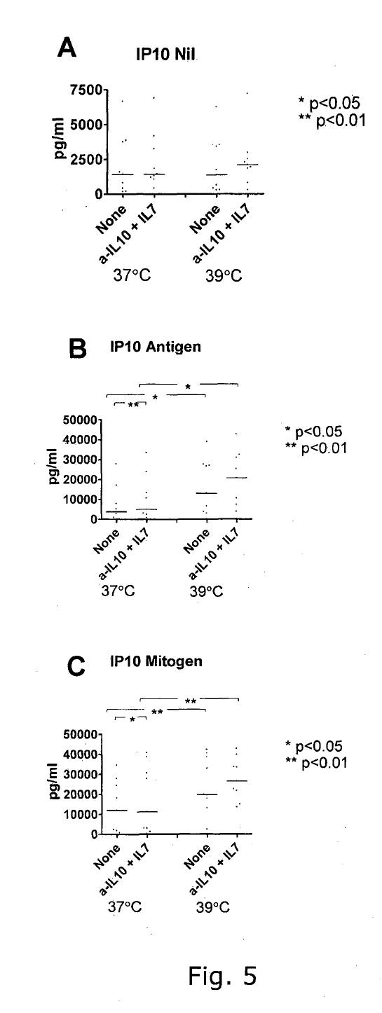

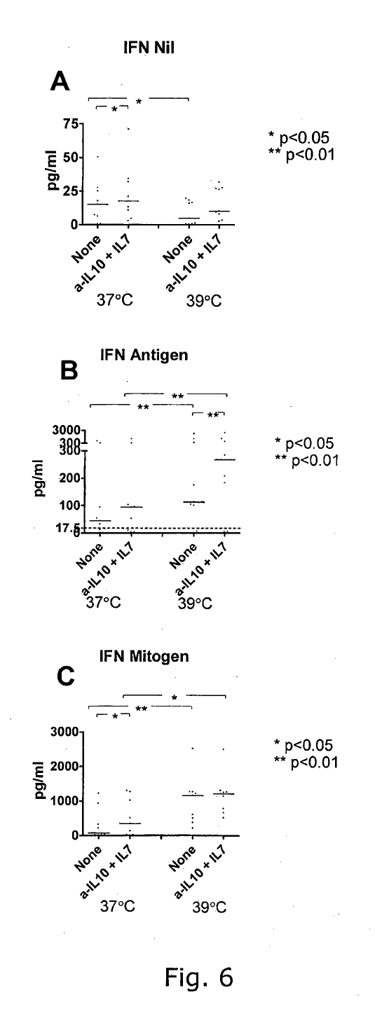

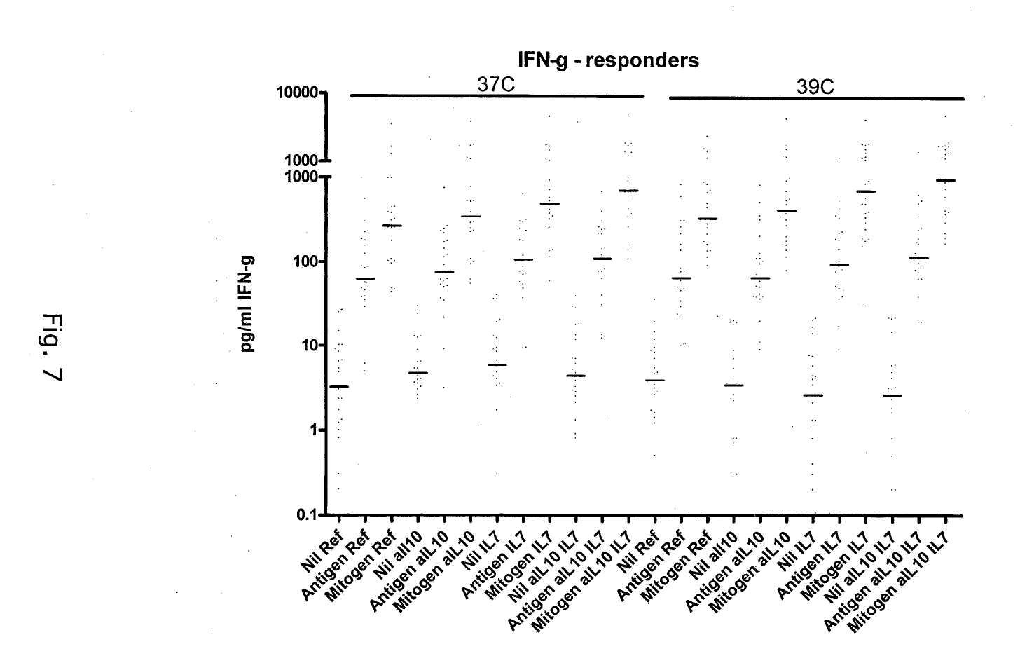

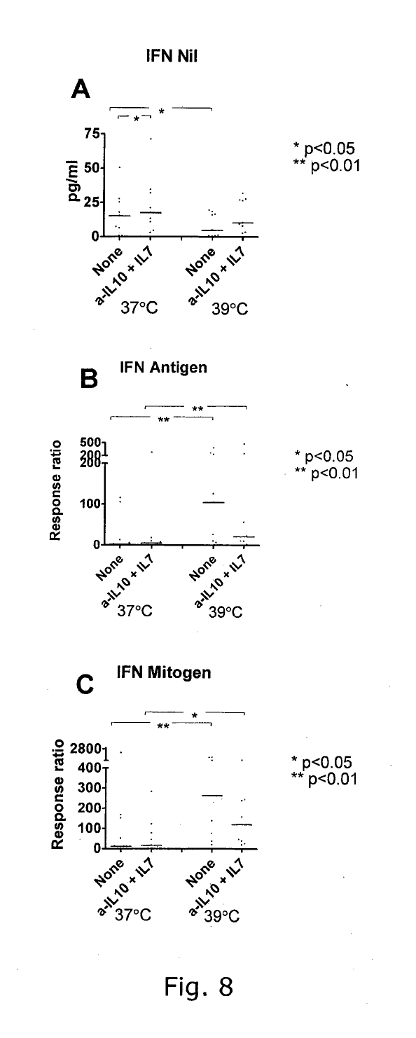

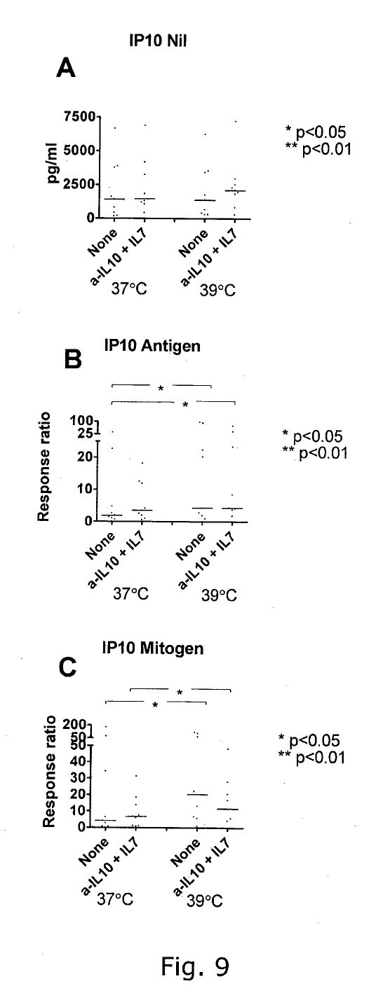

IP-10 responses to antigen and mitogen stimulation at 37° vs. 39° Celsius, and the influence of IL-7 addition and blockade of IL-10. Whole blood was drawn and incubated 18 h at 37° or 39° Celsius, in the presence or absence of IL-7, and blocking antibodies to IL-10. First four columns are experiments at 37° Celsius, the latter four at 39° Celsius.

3A presents data from 11 “TB10.4 non-responders” stimulated with overlapping peptides from the antigen TB10.4. Values are subtracted the levels in the unstimulated sample

3B presents data from 23 “TB10.4 responders” stimulated with overlapping peptides from the antigen TB10.4. Values are subtracted the levels in the unstimulated sample

3C presents the pooled background levels from responders and non-responders

3D presents the pooled mitogen stimulated levels from responders and non-responders. Values are subtracted the levels in the unstimulated sample

FIG. 4

IFN-γ responses to antigen and mitogen stimulation at 37° vs. 39° Celsius, and the influence of IL-7 addition and blockade of IL-10. Whole blood was drawn and incubated 18 h at 37° or 39° Celsius, in the presence or absence of IL-7, and blocking antibodies to IL-10. First 4 columns are experiments at 37° Celsius, the latter 4 at 39° Celsius.

4A presents data from 11 “TB10.4 non-responders” stimulated with overlapping peptides from the antigen TB10.4. Values are subtracted the levels in the unstimulated sample

4B presents data from 23 “TB10.4 responders” stimulated with overlapping peptides from the antigen TB10.4. Values are subtracted the levels in the unstimulated sample

4C presents the pooled background levels from responders and non-responders (the unstimulated levels)

4D presents the pooled mitogen stimulated levels from responders and non-responders. Values are subtracted the levels in the unstimulated sample.

FIG. 5

IP-10 responses from the QuantiFERON TB Gold In tube test tubes from 9 patients with culture confirmed TB. Whole blood was collected in the QuantiFERON TB Gold

In tube system and was incubated 18 h at 37 or 39° Celsius with or without addition of IL-7 and blockade of IL-10. Median levels for each column are depicted.

FIG. 6

IFN-γ responses from the QuantiFERON TB Gold In tube test tubes from 9 patient with culture confirmed TB. Whole blood was collected in the QuantiFERON TB Gold

In tube system and was incubated 18 h at 37 or 39° Celsius with or without addition of IL-7 and blockade of IL-10. Median levels for each column are depicted.

FIG. 7

IFN-γ responses in unstimulated samples (nil) and in response to TB10.4 antigen and mitogen stimulation at 37° vs. 39° Celsius, and the influence of IL-7 addition and blockade of IL-10 (example 2-5). Whole blood was drawn and incubated 24 h at 37° or 39° Celsius, in the presence or absence of IL-7 and blocking antibodies to IL-10. First 12 columns are experiments at 37° Celsius, the latter 12 at 39° Celsius.

FIG. 8

IFN-γ responses in unstimulated samples (nil) and in response to TB specific antigens and mitogen stimulation at 37° vs. 39° Celsius, and the influence of IL-7 addition and blockade of IL-10. Whole blood was drawn and incubated 24 h at 37° or 39° Celsius, in the presence or absence of IL-7 and blocking antibodies to IL-10. First 6 columns are experiments at 37° Celsius, the latter 6 at 39° Celsius.

FIG. 9 IP-10 responses in unstimulated samples (nil) and in response to TB specific antigens and mitogen stimulation at 37° vs. 39° Celsius, and the influence of IL-7 addition and blockade of IL-10. Whole blood was drawn and incubated 24 h at 37° or 39° Celsius, in the presence or absence of IL-7 and blocking antibodies to IL-10. First 6 columns are experiments at 37° Celsius, the latter 6 at 39° Celsius.

### REFERENCES

Waters W R, Nonnecke B J, Olsen S C, Palmer M V. Effects of pre-culture holding time and temperature on interferon-gamma responses in whole blood cultures from Mycobacterium bovis-infected cattle. Veterinary Microbiology 2007 119(2-4):277-282

Robbe-Austerman S, Krull A C, Stabel J R. Time delay, temperature effects and assessment of positive controls on whole blood for the gamma interferon ELISA to detect paratuberculosis. Journal of Veterinary Medicine. 2006;53(5):213-7.

Basu S, Srivastava PK. Fever-like temperature induces maturation of dendritic cells through induction of hsp90. International Immunology, 2003, 15(9): 1053-61

Huang Y H, Haegerstrand A, Frosteggrd 3. Effects of in vitro hyperthermia on proliferative responses and lymphocyte activity. Clinical Experimental Immunology, 1996,103:61-66

Kappel M, Diamant M, Hansen M B, Klokker M, Pedersen B K. Effects of in vitro hyperthermia on the proliferative response of blood mononuclear cell subsets, and detection of interleukins 1 and 6, tumour necrosis factor-alpha and interferon-gamma. Immunology. 1991 July;73(3):304-8.

Schiller I, Waters W R, Vordermeier H M, Nonnecke B, Welsh M, Keck N, Whelan A, Sigafoose T, Stamm C, Palmer M, Thacker T, Hardegger R, Marg-Haufe B, Raeber A, Oesch B. Optimization of a whole-blood gamma interferon assay for detection of Mycobacterium bovis-infected cattle. Clinical and Vaccine Immunology, 2009, 16(8): 1196-1202

Feske M, Nudelman R J, Medina M, Lew J, Singh M, Couturier J, Graviss E A, Lewis D E. Enhancement of human antigen-specific memory T-cell responses by interleukin-7 may improve accuracy in diagnosing tuberculosis. Clinical and Vaccine Immunology, 2008,19:1616-22

Denis M, Wedlock D N, McCarthy A R, Parlane N A, Cockle P3, Vordermeier H M, Hewinson R G, Buddle B M. Enhancement of the sensitivity of the whole-blood gamma interferon assay for diagnosis of Mycobacterium bovis infections in cattle. Clinical and Vaccine Immunology, 2007, 11: 1483-9

1. A method for generating a test-antigen specific cell-mediated immune response comprising the steps of;
a) providing a sample comprising cells of the immune system capable of generating a cell-mediated immune response from a mammal
b) incubating said sample at hyperthermic conditions with at least one test-antigen
c) determining the test-antigen specific cell-mediated immune response in said sample,
wherein said incubation at hyperthermic conditions generates an augmentation of the test-antigen specific immune response when compared to a reference level obtained by incubation under normal thermic conditions of 37° Celsius.

2. A method according to claim 1, wherein the test-antigen specific cell-mediated immune response is determined by measuring the level of at least one immune signalling molecule.

3. A method according to claim 2, wherein the immune signalling molecule is a cytokine or chemokine.

4. A method according to claim 2, wherein said immune signalling molecule level is determined by measuring the level of mRNA and/or protein.

5. A method according to claim 4, wherein said determination of the immune signalling molecule level is performed using a method selected from the group consisting of qPCR, RT-PCR, qRT-PCR, ELISA, ELISPOT Luminex, Multiplex, Immunoblotting, immunochromatographic lateral flow assays, Enzyme Multiplied Immunoassay Techniques, RAST test, Radioimmunoassays, immunofluorescence and various immunological dry stick assays.

6. A method according to claim 1, wherein said immune signalling molecule is selected from the group consisting of IP-10, INF-γ, IL-2, MIG, TNF-α, MIP-1 a, MCP-1, MCP-2, MCP-3, IL-1b, IL-RA, sIL-2R, and IL-12.

7. A method according to claim 6, wherein said immune signalling molecule is IP-10.

8. A method according to claim 6, wherein said immune signalling molecule is IFN-γ.

9. A method according to claim 1, wherein at least one immune-modulator selected from the group consisting of the cytokines IL-7, IL-15, 1L-21, neutralizing antibodies binding 1L-10, IL-4, IL-5, beads coated with anti-CD25 antibodies, beads coated with anti-CD39 antibodies, sense or antisense oligonucleotide to genetic material encoding IL-10, JAKl or TYK2, a CpG containing oligonucleotide, an oligonucleotide acting as a TLR modulating agent, and a TLR modulating agent is added in step b.

10. A method according to claim 1, wherein said at least one test-antigen is selected from the group comprising ESAT-6, CFP-10, TB7.7, and other RD-1 and RD-11 antigens.

11. A method according to claim 1, wherein said sample is whole blood or cells derived from blood, pleural fluid, bronchial fluid, tissue biopsies, ascites liquid, and/or cerebrospinal fluid.

12. A method according to claim 1, wherein said sample comprises cells selected from the group consisting of peripheral mononuclear cells, T cells, CD4 T cells, CD8 T cells, gamma-delta T cells, monocytes, macrophages, dendritic cells and NK cells.

13. A method according to claim 1, wherein said hyperthermic conditions is incubation at a temperature between 38.5-41.0° Celsius.

14. A method according to claim 1, wherein said hyperthermic conditions is incubation at a temperature between 39-40° Celsius.

15. A method according to claim 1, wherein sugar in the form of hydrocarbons and/or glycans is added in step b,

16. A method according to claim 1, wherein said method generates an improved signal to noise ratio of the test-antigen specific cell-mediated immune response,

17. A method according to claim 1, wherein said test-antigen specific cell-mediated immune response is used to diagnose an infection caused by a microorganism capable of expressing the said test antigen,

18. A method according to claim 1, wherein said test-antigen specific cell-mediated immune response is used to detect a vaccination response,

19. A method according to claim 1, wherein said test-antigen specific cell-mediated immune response is used to detect a cancer or neoplasm or malignancy.

20. A method according to claim 17, wherein said microorganism is selected from the group consisting of Mycobacteria, Leishmania, Chlamydia and Cytomegalovirus.

21. A method according to claim 20, wherein the Mycobacteria belongs to the M. tuberculosis complex organisms (M. tuberculosis, M. bovis and M. africanum), and Mycobacteria where the region of difference (RD1) has not been deleted (M. kansasii, Kszulgai, M. marinum, M. flavescens, M gastril) or Mycobacteria pathogenic to humans (M. avium, M. lepra or other non-tuberculous mycobacteria),

22. A method according to claim 21, wherein the Mycobacteria is M. tuberculosis.
